(19) United States
(12) Patent Application Publication
(10) Pub. No.: US 2009/0041838 A1
(43) Pub. Date: Feb. 12, 2009

(54) Anti-Misuse Microparticulate Oral Drug Form

(75) Inventors: Florence Guimberteau, Montussan (FR); Remi Meyrueix, Lyon (FR); Gerard Soula, Meyzieu (FR)

Correspondence Address: PATTON BOGGS LLP, 8484 WESTPARK DRIVE, SUITE 900, MCLEAN, VA, 22102, US

(73) Assignee: Flamel Technologies, S.A., Venissieux (Cedex) (FR)

(21) Appl. No.: 11/883,935

(22) PCT Filed: Feb. 8, 2006
(86) PCT No.: PCT/EP06/50784

§ 371 Date: Apr. 17, 2008

(30) Foreign Application Priority Data
Feb. 8, 2005 (FR) 05 50364

(51) Int. Cl. A61K 9/50; A61K 9/14; A61K 9/22

(52) U.S. Cl. 424/452; 424/490; 424/494; 424/493; 424/497; 424/465

(57) ABSTRACT

The invention relates to solid microparticulate oral dosage forms having a composition that prevents the misuse of the active pharmaceutical ingredient (API) contained therein. The aim of the invention is to prevent the improper use of solid oral drugs for any use other than the therapeutic use(s) officially approved by the appropriate public health authorities. Another aim of the invention is to provide novel analgesic drugs which can be used to: prevent the misuse of, and addiction to certain analgesics and/or to control plasma concentration variability and/or to facilitate oral; administration; and/or to combine analgesics with one another and/or with one or more active ingredients in the same oral form. More specifically, the invention relates to a solid oral drug form comprising anti-misuse means and at least one active ingredient, which is characterized in that: at least part of the active ingredient is contained in microparticles; and the anti-misuse means comprise anti-crushing means (a) which enable the microparticles of the active ingredient to resist crushing, such as to prevent the misuse thereof. According to the invention, the drug form can also comprise means (b) for preventing the misuse of the active ingredient following a possible liquid extraction process.

The field of the present invention is that of solid microparticulate oral drug forms, the composition of which makes it possible to prevent misuse of the active pharmaceutical ingredient (API) contained therein. In particular, the field of the present invention is that of analgesic solid microparticulate oral drug forms, the composition of which makes it possible, in particular, to reduce the number of daily doses taken, for analgesic purposes, and to prevent misuse of the analgesic active pharmaceutical ingredient (APIa) contained therein.

The active ingredients considered (APIs) are active pharmaceutical ingredients, for example those classified in the category of narcotic products. The latter are those of which abuse can give rise to drug addiction-related behavior. More particularly, the active ingredients considered are analgesic active ingredients (APIas).

For the purpose of the present disclosure, the expression “API” denotes both a single active ingredient and a mixture of several active ingredients. For the purpose of the present disclosure, the expression “APIa” denotes both a single analgesic active ingredient and a mixture of several active ingredients, at least one of which is an analgesic active ingredient.

For the purpose of the present invention, the term “microparticulate drug form” is intended to mean any form in which the API is contained in microparticles less than 1000 microns in size. These particles containing the API can be microcapsules for modified release of API. In the latter case, the microcapsules are, for example, coated with a polymer film which controls the rate of release of the API after oral administration.

The objective targeted by the present invention is to prevent the improper use of solid oral medicaments for any use other than the therapeutic use(s) officially approved by the appropriate public health authorities. In other words, it is a question of preventing the intentional or unintentional misuse of solid oral medicaments, in particular the case of APIs in the narcotics category and APIas.

### Situation of the Problem

Misuse is mainly encountered in the following cases:
(a) addictive behavior (drug addiction, doping),
(b) criminal behavior (chemical dependency),
(c) use of a medicament in a manner not in accordance with the medical recommendations (posology), inadvertently or due to disabilities affecting the patient,
(d) self-medication.

In case a), or even in case b), individuals who have the intention of misusing a solid oral medicament will generally apply themselves to making it either into a pulverulent form which can be inhaled or swallowed, or into a liquid form which can be injected using a syringe.

Obtaining of an injectable liquid form from a solid oral medicament involves a step consisting of aqueous or organic extraction of the API targeted. This extraction is generally preceded by crushing.

Methods of administration by inhalation or by injection are particularly suitable for drug addicts because they are methods which make it possible to accentuate the effects of the API and which promote its absorption in the body over short periods of time. When this powder is aspirated via the nose or dissolved in water and injected, the desired effects, doping effects or effects producing a feeling of euphoria, of the API manifest themselves very rapidly and in an exacerbated manner.

The misuse of solid oral medicaments can also be observed when the medicament is chewed before being swallowed, instead of being swallowed rapidly in accordance with the posology.

The risks associated with addictive behavior (a) and criminal behavior (b) and with self-medication (d) are obvious. It will be recalled that the misuse of medicaments by injection is a serious situation: the excipients can be responsible for local tissue necroses, for infections, and for respiratory and cardiac problems.

As regards abuses (c) of the use of a medicament related to inattention and/or to disabilities of the patient, they can also have serious consequences. For example, the chewing of modified-release forms of API before swallowing converts the medicament into an immediate-release form. Thus, at best, the medicament is ineffective after a very short period of time, and, at worst, it becomes toxic.

As regards analgesic active ingredients, their use poses several major public health problems. The first problem (P1) is that a large number of analgesics are also narcotic products which induce dependency in patients. This dependency is in particular accentuated when the plasma concentration profile for the APIa exhibits very pronounced peaks and troughs. It would therefore be very advantageous to have a modified-release form which makes it possible to obtain a plasma concentration profile in the form of a “plateau” which levels out the peaks and troughs phenomena.

The second problem (P2) is related to the fact that certain immediate-release oral pharmaceutical forms of APIa (IR forms) produce erratic plasma profiles and do not guarantee an analgesic action which is homogeneous, effective and tolerable for all patients. In this way, some patients are incorrectly treated and/or, even more seriously, are victims of dangerous side effects. This great variability with premature and massive release of APIa can have serious consequences. Firstly, the patients for whom the concentration peak is early and of very large amplitude are victims of overdoses which can be fatal. Secondly, the early decrease in plasma concentration after the peak is reflected by a very low APIa concentration level at the end of the period between two administrations. Thus, after having been subjected to an overconcentration of APIa corresponding to the peak, the patients are insufficiently treated at the end of the period between two administrations. They are no longer under the effect of the APIa and therefore suffer from pain. Thirdly, this great variability leads the practioner to limit the prescribed doses and certain patients can be incorrectly treated.

It would therefore be an advantage to have oral pharmaceutical forms of APIa which make it possible to control the plasma concentration (in particular, the maximum plasma concentration: Cmax) so as to avoid any massive and/or early and/or rapid release of the APIa.

The third problem (P3) is that of making it easier to administer an APIa-based medicament orally to populations who have trouble swallowing and are incapable of swallowing large tablets: infants, children, elderly individuals or patients suffering from highly incapacitating diseases such as cancers. It is clear that these difficulties in oral administration have a detrimental effect on whether patients adhere to their treatment. Now, to date, the only known suitable oral form consists of sachets of powder to be dispersed in a liquid. It would therefore be advantageous to have an oral form that is more convenient to use.

The fourth problem (P4) is that of the combination of several APIas with one another, or even with other non-analgesic API active ingredients in the same pharmaceutical form. These combinations, which are sometimes useful in therapeutic terms, can sometimes be made difficult due to the chemical incompatibility (degradation) between two active ingredients and/or due to the need to have distinct release kinetics for the various APIas or APIs in modified-release forms.

The fifth problem (P5) is related to the fact that analgesics, in particular morphine derivatives, are often the subject of misuse. This misuse is intentional or unintentional improper use of APIa-based solid oral medicaments for any uses other than the therapeutic use(s) officially approved by the appropriate public health authorities. Various forms of misuse, which also apply to analgesic active ingredients, have been recalled above in (a), (b), (c) and (d).

There clearly exists, therefore, a serious public health problem related to the misuse of medicaments, and in particular of solid oral medicaments, and more especially in the case of analgesic active ingredients.

This growing phenomenon is increasingly worrying to the health authorities, which are multiplying appeals for the development of drug forms for preventing improper use.

### PRIOR ART

To the applicant's knowledge, the only attempts to solve the problem of misuse have consisted in combining with the API, for example an opioide, an antagonist which counteracts the physiological effects of the opioide API, when the medicament is improperly used.

This pseudosolution has certain dangers for users, in particular during use under approved conditions. Furthermore, combinations of API and of other active compounds such as antagonists of the API, are difficult to control and pose a serious public health problem. There is a risk of the therapeutic effect being hindered or even destroyed. In addition, these proposals are not able to block all the pathways to improper use.

Patent U.S. Pat. No. 6,696,088 reports a multiparticulate oral pharmaceutical form, indicated as being resistant to misuse. The latter comprises particles of opioide agonist API in a modified-release form and particles comprising an antagonist of the opioide. The form containing the antagonist is described as releasing less than 36%, and even more preferably less than 6.2% of the antagonist API over a period of 36 h. The two types of particles are interdispersed.

The fact that, when a misuse is carried out, the microparticles are crushed in order to extract them from the opioide API results in the immediate and concomitant release of API and its antagonist and thus a limitation of the desired effects of the improperly used opioide.

This invention is based on the use of an active substance other than the API and does not propose a solution for decreasing the impact of the crushing or reducing the extraction of the API.

Patent application WO 2004/054542 describes a semi-liquid oral pharmaceutical form. It is in the form of a gel capsule (for example of gelatin) comprising the API in a matrical phase composed of a water-insoluble highly viscous liquid (sucrose acetate isobutyrate) and of a polymer (cellulose acetate butyrate) that is supposed to form a network in the liquid phase. The formulation can optionally comprise a compound for modifying the rheology of the pharmaceutical form and a solvent. By adjusting the various compounds and concentration of the formulation, the authors state that they are able to modify the plasma profiles of the API (oxycodone base) administered to dogs.

This reference does not provide any solution for blocking misuse by injection. In addition, its viscosity drops greatly when small amounts of ethanol are added.

Patent application WO 2004/056337 reiterates the invention detailed in patent application WO 2004/054542. The pharmaceutical form comprises one or more APIs that can be released immediately for the fraction of API that is in the gelatin envelope, and in a controlled manner for the fraction of APIs that are in the core (liquid, gel or solid). The APIs present are, in the examples, an opioide agonist (oxycodone) and an opioide antagonist (naltrexone).

The system proposed is macroscopic and the active ingredient can be recovered by simple cutting up. It should also be noted that it contains an antagonist.

Patent application US 2003/0068371 describes an oral pharmaceutical formulation comprising an opiate API (oxycodone), an antagonist of this API (naloxone) and a gelling agent (e.g. xanthan gum). In particular, this US application discloses granules of API comprising lactose, xanthan gum, povidone and an overcoating based on Eudragit RS 30D®/triacetin/antagonist. The gelling agent is presented as conferring a viscosity on the formulation such that it cannot be nasally and parenterally administrable. This answer is not sufficient since, in accordance with this invention, the use of an antagonist is obligatory. Now, as already mentioned, the presence of the antagonist is a major drawback with regard to the medical risks possibly run by the users and the risks of inhibition of the targeted therapeutic effect. Finally, this formulation does not comprise any anticrushing means, can therefore be made into a pulverulent form and, consequently, can be the subject of misuse by nasal or oral administration.

Patent application EP-A 0 647 448 discloses a solid oral pharmaceutical form for the release of an opioide APIa (morphine) over a period of at least 24 h. The APIa is contained in microparticles between 0.1 and 3 mm in size. These microparticles can each be formed by a matricial substrate containing the APIa and a hydrophobic compound. According to a variant, the microparticles are microcapsules of reservoir type and are each formed by a nucleus comprising an inert core (sugar), coated with a layer comprising APIa and excipients (lactose/vinylpyrrolidone/hydroxypropyl-methylcellulose (HPMC)) and with a coating for controlling the release of the APIa. This coating comprises, for example, a methacrylic copolymer (Eudragit® RS 30D/triethyl citrate/talc). An overcoating (APIa/HPMC) can be envisioned. These microcapsules gradually release the APIa over 24 h in an in vitro dissolution test, at 37° C. and at gastric pH.

Patent U.S. Pat. No. 6,627,635 describes a pharmaceutical form for sustained release over a period of 12 to 24 h, containing an opioide agonist (hydrocodone) and an antagonist (naltrexone) of the opioide, as anti-misuse means. This pharmaceutical form can be of matrix or reservoir type (APIa nucleus+coating controlling the diffusion of the APIa). It can be in the form of a tablet or of microparticles. The latter have a diameter of between 100 and 2500 μm (500-2000 μm). The coating is, for example, based on ethylcellulose and/or on a methacrylic copolymer (Eudragit® RS 30 D and/or RL 30 D), and on an optional plasticizer (triethyl citrate). HPMC can be used in the coating or in an overcoating.

The solid oral pharmaceutical forms according to patent application EP 0647448 and patent U.S. Pat. No. 6,627,635 do not disclose gallenic means for solving the abovementioned problems P1 to P5. In particular, patent application EP 0647448 does not describe anti-misuse means—e.g. anti-crushing means—(problem P5). The anti-misuse means according to patent U.S. Pat. No. 6,627,635—antagonist—are absolutely not satisfactory. This is because the antagonists of the APIa are pharmaceutically active substances which are therefore potentially dangerous for users, and which can act against the normal use of the medicament.

### OBJECTIVES OF THE INVENTION

Under these circumstances, one of the essential objectives of the present invention is to make up for the deficiencies of the prior art.

Another essential objective of the invention is to provide new solid oral medicaments, the misuse of which will be made very difficult, or even impossible, in particular for the abovementioned cases (a), (b), (c) and (d), preferably without resorting to substances, other than the API, that may be pharmaceutically active and therefore dangerous for users, or may even inhibit the API, for instance antagonists of the API.

Another essential objective of the invention is to provide a new solid oral medicament which makes it possible to avoid fraudulent abuse of the properties of the API that it contains, by preventing any conversion of the medicament which would make it possible to take it orally, nasally and/or by injection (intravenous, subcutaneous, intramuscular, etc.) outside the therapeutic context. In so doing, the risks associated with these abuses would be prevented or, at the very least, greatly reduced.

Another essential objective of the invention is to provide a new solid oral medicament which makes it possible to avoid misuse, while at the same time guaranteeing, for the patient normally followed up, a quality of treatment, in particular a dose, in accordance with said patient's needs.

Another essential objective of the invention is to provide a new solid oral medicament which makes it possible to avoid misuse, without affecting the pharmacological properties of the medicament, and without causing the patient using the medicament normally, to run any additional risks, and finally, without being detrimental to the patient's comfort during administration.

Another essential objective of the invention is to provide new solid oral analgesic medicaments which allow, at the same time:
prevention of misuse, the latter being made very difficult or even impossible, in particular for the abovementioned cases (a), (b), (c) and (d), preferably without resorting to antagonists of the APIas,
modified release of an APIa according to a plasma concentration profile in the form of a “plateau”, which levels out the peaks and troughs phenomena, and thus provides an advantageous solution to the major public health problem represented by addiction to certain APIas;
and/or control of the variability of the plasma concentration (in particular the maximum plasma concentration: Cmax) so as to prevent a high inter- and/or intraindividual variability in the quality of the treatment;
and/or facilitation of administration for populations incapable of swallowing tablets which are often considerable in size, namely: infants, children and elderly individuals;
and/or the combination of several APIas with one another, or even with other non-analgesic API active ingredients in the same pharmaceutical form, even in the event of incompatibility between the substances in question and/or when the various APIs and/or APIas must have distinct release kinetics;
and/or the provision of an oral pharmaceutical form of APIa which can be administered one or more times a day and which offers the possibility of mixing the APIa with one or more active ingredients in the same oral form, with the possibility of readily and independently adjusting the release times of the various active ingredients.

Another essential objective of the invention is to provide an oral pharmaceutical form of APIa which is used in such a way that it provides a quality of treatment which is more uniform and more reproducible from one patient to the other, compared with that which is proposed in the prior art.

Another essential objective of the present invention is to provide a means for reducing the inter- and/or intraindividual standard deviation of the maximum concentration Cmaxof the plasma concentration profile.

Another essential objective of the invention is to provide an oral pharmaceutical form of APIa which reduces the inter- and/or intraindividual variability of the in vivo absorption of the APIa, which is a direct consequence of the sensitivity of certain modified-release oral gallenic forms (gastroretentive tablets, for example) with respect to the inter- and/or intraindividual variability of gastric emptying.

Another essential objective of the invention is to provide an oral pharmaceutical form of APIa which can be administered once or twice a day and is at least as effective as the immediate-release once-a-day forms currently in use.

Another essential objective of the invention is to provide an oral pharmaceutical form of APIa which exhibits an in vitro dissolution profile independent of the dose of APIa.

Another essential objective of the invention is to provide an oral pharmaceutical form of APIa, for which the microparticles of which it is composed have the same composition by weight irrespective of the therapeutic dose of APIa targeted.

Another essential objective of the invention is to provide an oral pharmaceutical form of APIa which can be administered once a day and which limits the risk of tissue deterioration due to local overconcentration of APIa.

Another essential objective of the invention is to provide an oral pharmaceutical form of APIa which can be administered once a day and which, despite the variability in solubility of the APIa in water as a function of the pH, releases the APIa according to the same kinetics, whether or not the patient is fasting.

Another essential objective of the invention is to provide an oral pharmaceutical form of APIa which can exist in various pharmaceutical presentation forms, including in particular: tablet, sachet, oral suspension, gel capsule, etc.

Another essential objective of the invention is to provide a new solid oral medicament which makes it possible to prevent fraudulent abuse of the properties of the API that it contains, by preventing any conversion of the medicament which would make it possible to take it orally, nasally and/or by injection (intravenous, subcutaneous, intramuscular, etc.) outside the therapeutic context. In so doing, the risks associated with these abuses will be prevented or, at the very least, greatly reduced.

Another essential objective of the invention is to provide a new solid oral medicament which makes it possible to avoid misuse, while at the same time guaranteeing, for the patient normally followed up, a quality of treatment, in particular a dose, in accordance with said patient's needs.

Another essential objective of the invention is to provide a new solid oral medicament which makes it possible to avoid misuse, without affecting the pharmacological properties of the medicament, and without causing the patient using the medicament normally, to run any additional risks, and finally, without being detrimental to the patient's comfort during administration.

Another essential objective of the invention is to provide a new solid oral medicament which makes it possible to avoid misuse, which is simple to obtain, and for which the method of production does not cause its cost price to increase.

### BRIEF DESCRIPTION OF THE INVENTION

In order to attain these objectives, it is to the inventors' credit to have reformulated the general problem of the misuse of pharmaceutical forms.

If one examines the various illicit methods of administration of an active ingredient, it appears in fact that crushing of the dry form is an obligatory step.

In the case of misuse by nasal administration, the dry pharmaceutical form must, beforehand, be converted into a pulverulent powder which can be aspirated. Crushing of the pharmaceutical form is therefore clearly an obligatory step.

In the case of misuse by oral administration of a sustained-release dry form, it is necessary to accelerate the release of the active ingredient by finely crushing the microparticles or the tablet.

In the case of misuse by parenteral administration, it is necessary, beforehand, to extract the API in a liquid phase, in practice water or organic solvents, at a concentration sufficiently high to avoid injecting volumes which are too large, for example greater than 1 ml. This extraction step is facilitated by a prior step of crushing the dry form in order to allow dissolution or suspension of the active ingredient. In addition, at the end of this extraction phase, the misuse is only possible if the viscosity of the liquid is not too high (for example, less than or equal to 100 mPa·s).

Thus, the crushing of a dry form is also an obligatory step for the misuse of said pharmaceutical form by parenteral administration.

It is therefore to the applicant's credit to have reformulated the problem of combating the misuse of dry pharmaceutical forms, by distinguishing:
a main problem of preventing the crushing of the system containing the API,
and a secondary problem of preventing the misuse of the API after it has possibly been extracted.

This novel approach has allowed the applicant to discover, surprisingly and unexpectedly, that it is advisable to involve, in the composition of the medicament the misuse of which it is sought to prevent, the API in the form of microparticles and a combination of pharmaceutically acceptable excipients, which may or may not be in microparticulate form, and the method of physicochemical action of which makes it possible to act against, or even to make impossible, any intentional or unintentional act of misuse.

Thus, the invention relates, in the main respect, to a solid oral drug form, characterized in that at least a part of the API that it comprises is contained in microparticles, and in that it also comprises anti-crushing means (a) provided so as to allow the microparticles of API to withstand crushing, so as to prevent misuse. In other words, the invention relates to a solid oral drug form comprising anti-misuse means and at least one active ingredient (API), characterized in that at least a part of the API is contained in microparticles, and characterized in that the anti-misuse means comprise anti-crushing means (a) provided so as to allow the microparticles of API to withstand crushing, so as to prevent misuse.

The drug form according to the invention solves in particular the main stated problem and fulfils the set objectives in an effective, simple and economical manner, using physicochemical means. The latter are completely harmless to the normal user. They are pharmacologically neutral (inert) compounds approved by the pharmacopeia and by the public health authorities responsible for granting marketing authorizations for medicaments.

Moreover, in order to attain the targeted objectives, it is to the inventors' credit to have combined means for sustained release of APIa, capable of solving at least one of problems P1 to P4, and specific anti-misuse means, i.e. anti-crushing means, capable of solving problem P5.

This combination did not go without saying. It was in fact necessary to develop means for controlling the release of the APIa which are compatible with the anti-misuse means, taking into account the fact that the crushing of a dry form is also an obligatory step for the misuse of said pharmaceutical form by parenteral administration.

Thus, the invention also relates to an oral drug form comprising anti-misuse means and a plurality of microcapsules for modified release of at least one analgesic active ingredient (APIa), at least a part of said microcapsules consisting individually of a nucleus comprising at least one APIa and coated with at least one coating for the modified release of the APIa; the average diameter of said microcapsules being less than or equal to 1000 μm, preferably between 50 and 800 μm, more preferably between 50 and 600 μm, and even more preferably between 80 and 400 μm; characterized:
in that it comprises at least 1000 microcapsules per dose;
and in that the amount of APIa and the modified-release coating are such that they allow an administration once or twice a day for analgesic purposes.

The drug form according to the invention solves in particular the stated problem (P1), i.e. that of the dependency of consumers with respect to the APIas, along with the incidental, but no less important, problem (P5) of misuse.

It fulfils the set objectives in an effective, simple and economical manner using physicochemical means: use of coated APIa microcapsules and misuse means which are compatible.

All these elements are completely inoffensive to the normal user. They are pharmacologically neutral (inert) compounds approved by the pharmacopeia and by the public health authorities responsible for granting marketing authorizations for medicaments.

Preferably, the drug form according to the invention also comprises means (b) provided for preventing misuse of the API after a possible liquid extraction.

Preferably, the drug form according to the invention is characterized in that it is free of antagonist(s) of the API.

### DETAILED DESCRIPTION OF THE INVENTION

According to a preferred embodiment, the invention relates to a solid oral drug form, characterized in that at least a part of the API that it comprises is contained in microparticles, and in that it comprises, firstly, means (a) for preventing, or at the very least making very difficult, the crushing of the microparticles containing the API, and secondly, means (b) for making very difficult the misuse of the API after a possible liquid extraction carried out for the purposes of misuse.

Preferably, the anti-crushing means (a) are:
an overcoating for protection of the microparticles of API having at least one of the following characteristics:
   viscoelastic properties for absorbing the energy dissipated during crushing,
   a low cohesive strength for promoting breaking of the overcoating and not of the microparticles,
   a low surface energy for promoting slipping of the microparticles during crushing,
   an ability to form a paste under high shear,
and/or excipients in the free state, i.e. not contained in nor supported by microparticles, and capable of acting against, or even preventing, the crushing of the microparticles of API.

The preferred anti-crushing means (a) are particles coated with an overcoating having specific physicochemical properties.

According to a specific embodiment of the drug form according to the invention, at least a part of the microparticles of API are microparticles, preferably microcapsules, for modified release of API.

Notably, the microparticles of API have an average diameter of less than or equal to 1000 μm, preferably between 50 and 800 microns, and more preferably between 100 and 600 microns.

According to a particularly advantageous arrangement of the invention concerning the case where the drug form comprises microparticles for modified release of API, the overcoating for protection of said microparticles is designed in such a way that it makes it possible, in the event of crushing, to maintain a non-immediate release for at least a part of said microparticles for modified release.

Advantageously, the protective overcoating represents, for example, between 1% and 60%, preferably between 10% and 60% by weight of the total mass of the microparticles containing the API.

Described hereinafter is a preferred embodiment of the drug form according to the invention, in which the overcoating for protection of the microparticles of API comprises:

(i) at least one film-forming compound which ensures the cohesion of the overcoating and at least one of the following three compounds:
(ii) a lubricant/caking agent,
(iii) a viscoelastic compound,
(iv) a plasticizer.

The film-forming compound (i) has the role of ensuring the cohesion of the overcoating. The film-forming compound (i) is, for example, chosen from:
cellulose derivatives,
acrylic derivatives,
and mixtures thereof.

The lubricant/caking agent (ii) is selected such that, under shear, it is capable of converting the solid drug form into a system on which the crushing has no or little hold. The lubricant/caking agent (ii) is preferably chosen from the group comprising:
stearic acid and stearates, preferably calcium stearate, zinc stearate or magnesium stearate;
magnesium oxide;
poloxamers;
sodium benzoate;
anionic, cationic or nonionic surfactants;
starches, preferably corn starch;
talc;
colloidal silica;
waxes, preferably hydrogenated plant oils, and even more preferably hydrogenated cotton seed oils, hydrogenated soybean oils, hydrogenated palm oils, hydrogenated castor oils; glyceryl behenates, tristearins, tripalmitins, trimyristins, yellow waxes, hard fats, anhydrous dairy fats, lanolins, glyceryl palmitostearates, glyceryl stearates, lauric acid macrogol glycerides, cetyl alcohols, glyceryl diisostearates, diethylene glycol monostearates, ethylene monostearates, omegas 3, and mixtures thereof;
fatty bases for suppositories comprising glycerol, triglycerides, theobroma oils, cocoa butters and mixtures thereof;
and mixtures thereof.

The role of the lubricant/caking agent (ii) is to greatly limit, or even eliminate, the abrasion of the microparticles containing the API when they are mechanically crushed. The lubricant (or slip agent)/caking agent (ii) makes it difficult to crush the multiparticulate drug form by facilitating its flow, thus reducing the shear stress applied to the product. The advantage of the lubricant (or slip agent)/caking agent (ii) to generate wall slip; the product does not therefore adhere to the wall of the mill, thereby preventing transmission of the shear stress to the active ingredient present in the microparticles.

The role of the viscoelastic agent (iii) is to dissipate the mechanical shear energy in order to protect the microparticles of API. This viscoelastic agent (iii) is, for example, selected from the group of following products:
poly-N-vinylamides,
gum bases,
fatty alcohols,
poly-N-vinyllactams,
polyvinyl alcohols (PVAs),
polyoxyethylenes (POEs),
polyethylene glycols (PEGs),
polydextroses,
hydrogenated mono-, di- and polysaccharides,
polyvinylpyrrolidones (PVPs) (the latter being preferred),
and mixtures thereof.

The role of the plasticizer (iv) is to increase the breaking strength of the overcoating. The plasticizer (iv) is preferably selected from the group of following products:
glycerol and its esters, preferably from the following subgroup: acetylated glycerides, glyceryl monostearate, glyceryl triacetate, glyceryl tributyrate,
phthalates, preferably from the following subgroup: dibutyl phthalate, diethyl phthalate, dimethyl phthalate, dioctyl phthalate,
citrates, preferably from the following subgroup, acetyl tributyl citrate, acetyl triethyl citrate, tributyl citrate, triethyl citrate,
sebacates, preferably from the following subgroup: diethyl sebacate, dibutyl sebacate,
adipates,
azelates,
benzoates,
plant oils, preferably cottonseed oils, soybean oils, palm oils, castor oils, and mixtures thereof;
fumarates, preferably diethyl fumarate,
malates, preferably diethyl malate,
oxalates, preferably diethyl oxalate,
succinates, preferably dibutyl succinate,
butyrates,
cetyl alcohol esters,
triacetin,
malonates, preferably diethyl malonate,
and mixtures thereof.

According to a variant, the excipients included in the anti-crushing means (a) can be chosen from:
compression agents,
and/or inert microbeads,
and/or gum bases,
and/or viscoelastic agents such as the viscoelastic agents (iii) defined above.

The inert microbeads are advantageously insoluble in an aqueous or aqueous-alcoholic medium and are uncompressible. These neutral beads support part of the crushing stresses, thus protecting the microparticles containing the API. They therefore render attempts at mechanical crushing ineffective.

In order to prevent the other prerequisite from misuse, namely the liquid extraction of the API, it is proposed, in accordance with an embodiment of the invention, to use, in the drug form, means (b) which make it possible to increase the viscosity of the liquid beyond 100, preferably 200, and even more preferably beyond 500 mPa·s, and even better still 1000 mPa·s.

Preferably, the means (b) provided for preventing the misuse of the API after a possible liquid extraction comprise “viscosity-modifying” excipients capable of increasing the viscosity of the extraction liquid so as to act against the misuse, in particular by injection.

Advantageously, the “viscosity-modifying” excipients capable of increasing the viscosity of the extraction liquid so as to act against the misuse, in particular by injection, are present:
in and/or on microparticles,
and/or in an overcoating of all or part of the microparticles of API,
and/or in the free state, i.e. not contained in nor carried by microparticles.

According to a preferred method, the “viscosity-modifying” excipients are capable of increasing the viscosity of the liquid used for the possible extraction according to kinetics similar to the kinetics of extraction of the API contained in the microparticles, so as to trap the extracted API in the viscous medium.

It is also to the applicant's credit to propose viscosity-modifying means (b) which are effective both in the case of an extraction in an aqueous phase or an extraction in an organic solvent.

For the purpose of the invention, the expression “similar kinetics” means that the kinetics of the increase in viscosity induced by the means (b) is, for example, substantially equal to 0.2-5 times, preferably to 0.3-3 times, and even more preferably to 0.3-2 times the kinetics of extraction of the API contained in the microparticles. In fact:
if the increase in viscosity is too rapid, it is possible to extract the microparticles of API which are still loaded;
if, on the other hand, the viscosity modification is too slow, it is possible to release the API into the liquid phase and to recover it before the viscosity modification has taken place.

Thus, the excipients included in the means (b) are preferably chosen from the groups of following polymers:
polyacrylic acids and their derivatives, and/or polyoxyethylenes (POEs), and/or
polyvinyl alcohols (PVAs),
polyvinylpyrrolidones (PVPs), and/or
gelatins, and/or
cellulose derivatives (e.g. hydroxypropylmethyl-cellulose, methylcellulose, hydroxyethylcellulose, carboxymethylcellulose), and/or
polysaccharides, preferably from the subgroup comprising: sodium alginate, pectins, guars, xanthans, carragheenans, gellans,
and mixtures thereof.

Advantageously, the excipients constituting the means (b) are mixtures of hydrophilic compounds and of hydrophobic compounds, so as to ensure a high viscosity (greater than 100 mPa·s, for example) of the extraction liquid, irrespective of whether it is aqueous or organic.

Preferably, the viscosity-modifying means (b) are in the form of microparticles. Even more preferably, these viscosity-modifying means (b) microparticles are physically indiscernible from the microparticles of API, in order to prevent them being sorted by an appropriate physical means. The microparticles comprising viscosity-modifying means (b) are indiscernible from the microparticles of API, in particular because they are the same size and/or of the same density and/or of the same shape and/or of the same color.

As regards the amount of viscosity-modifying excipients included in the means (b), it can be readily determined by those skilled in the art. This amount corresponds to the minimum amount required to modify the viscosity of 10 ml of extraction liquid to a value greater than or equal to 100 mPa·s.

According to another notable embodiment of the invention, the drug form comprises:
A. microparticles of API comprising means (a),
B. and microparticles of viscosity-modifying excipients included in the means (b).

The proportions A-B can be determined by those skilled in the art according to the desired therapeutic dose.

The microparticles containing the API can be microparticles for modified release of API, i.e. microparticles coated with a polymer film deposited according to the techniques known to those skilled in the art. On this question, the article “formes pharmaceutiques nouvelles” [New pharmaceutical forms] by Buri, Puisieux, Doelker and Benoit, Lavoisier 1985, p 175-227, will for example by consulted.

By way of examples of microparticles for modified release of API, mention may be made of those described in the following patent documents: EP-B-0 709 087 and WO-A-03/030878.

According to a first variant, the drug form according to the invention cannot be converted into a dry form which can be administered by nasal aspiration.

According to a second variant, the drug form according to the invention cannot be converted into an injectable form.

According to a third variant, the drug form according to the invention comprises immediate-release API and/or modified-release API.

According to a fourth variant, in the drug form according to the invention, extraction of the API by chewing and/or crushing is not effective.

Naturally, any combination of at least two of these four variants is included in the present invention.

Preferably, as regards a drug form comprising at least one APIa, said form makes it possible to obtain, after taking one dose, a plasma profile defined as follows:
Cmax/C18h≦Cmax*/C18h* preferably 1.5×Cmax/C18h≦Cmax*/C18h* and even more preferably 2.0×Cmax/C18h≦Cmax*/C18h* with
C18h representing the plasma concentration of APIa, 18h after taking the dose,
C18h* representing the plasma concentration of APIa obtained under the same conditions as C18h, with a reference immediate-release oral pharmaceutical form, containing the same dose of APIa,
Cmax representing the maximum plasma concentration of APIa after taking the dose,
Cmax* representing the maximum plasma concentration of APIa obtained under the same conditions as Cmax, with a reference immediate-release oral pharmaceutical form, containing the same dose of APIa.

Preferably, this drug form comprising an APIa is designed in such a way that it, and in particular the coating of the microcapsules, results in a decrease in the inter- and/or intraindividual standard deviation of the Cmax, when it is administered orally to a sample of individuals, whatever the fed state or fasting state of the individuals, compared with a pharmaceutical form for immediate release of APIa administered to this same sample of individuals, at the same dose, which makes it possible to ensure a smaller variability in effectiveness and in therapeutic safety of the pharmaceutical form.

One of the characteristics of the drug form according to the invention is thus defined through a reference clinical test in which the form is administered orally to a sample of human individuals, under experimental conditions which may, for example, be as follows: administration of the form (gel capsule or tablet or suspension) once a day, at a given dose, after breakfast, to 20 normal volunteers in the course of a crossover trial study. The plasma concentrations of APIa are measured at times: 0-0.25-0.5-0.75-1-1.5-2-3-4-6-8-10-12-16-18-20-24-36-48 hours post-administration.

This clinical test defines the invention in terms of the pharmacokinetics properties obtained specifically under the conditions of the test. Nevertheless, the invention is not limited to an implementation under the conditions of this reference clinical test.

The factor f of decrease in the inter- and/or intraindividual standard deviation of the Cmaxis defined as being the ratio of the inter- and/or intraindividual standard deviation of the Cmaxof the reference immediate-release pharmaceutical form, to the inter- and intraindividual standard deviation of the Cmax of the pharmaceutical form according to the invention, administered at the same dose of APIa.

Advantageously, the factor (f) of decrease in the inter- and/or intraindividual standard deviation of the Cmax is defined as follows: f≧1.05; preferably, f≧1.5, and even more preferably, f is between 2.0 and 20.

For the purpose of the present invention, the mean peak/trough modulation—PTM—of the plasma profile of an API is defined in the following way: on each of the individual plasma profiles, the individual maximum concentration cmax′ and the concentration cT′ are measured, T hours after a single oral administration. The PTM is the arithmetic mean of the cmax′/cT′ individual ratios.

For a product intended to be administered daily to the patient, T is 24 hours after the single administration. If the concentration cT′ (T=24 h) is below the limit of detection of the assay method used and below the limited detection of the method recommended by the pharmacopeia of the United States of America and/or known to those skilled in the art, the concentration c24′ used to calculate the PTM will be replaced with the concentration cx′ measured x hours after oral administration, x being the most belated hour at which it is possible to measure a concentration above the limit of detection of the method used. In this case, x is less than 24 hours after single administration. For example, x is equal to 18 h, or, failing this, 12 h.

For a product intended to be administered twice daily to the patient, T is 12 hours in single administration. Here also, if the concentration cT′ (T=12 h) is below the limit of detection of the assay method used and below the limit of detection of the method recommended by the pharmacopeia of the United States of America and/or known to those skilled in the art, the concentration c12′ used to calculate the PTM will be replaced with the concentration cx′ measured x hours after oral administration, x being the most belated hour at which it is possible to measure a concentration above the limit of detection of the method used. In this case, x is less than 12 hours after single administration.

The drug form according to the invention is designed in such a way that it results, when it is administered orally to a sample of individuals, in a mean peak/trough modulation of the plasma profiles of the APIa less than or equal to the mean peak/trough modulation of the APIa of the same sample of individuals having received the same dose of a form for immediate release of APIa.

For the purpose of the invention, the reduction in the peak/trough modulation of the plasma concentration profiles is given, for example, by the peak/trough modulation decrease factor g. The factor g is defined by the ratio of the peak/trough modulation of the reference immediate-release form to the peak/trough modulation of the form involved in the use according to the invention.

Preferably, the peak/trough modulation decrease factor g is such that: g≧1.05; preferably, g≧1.5, and more preferably, g is between 2.5 and 20.

In accordance with the use according to the invention, the coating or the matrix of the pharmaceutical form is designed in such a way that the oral administration of this form, to a sample of individuals, results in a variability of the peak/trough modulation of the plasma profiles of the API which is less than the variability of the peak/trough modulation of the API of the same sample of individuals having received the same dose of a form for immediate release of API.

For the purpose of the invention, the reduction in the variability of the peak/trough modulation of the plasma concentration profiles is given, for example, by the factor g′ for decrease in the standard deviation of the peak/trough modulation. The factor g′ is defined by the ratio of the standard deviation of the peak/trough modulation of the reference immediate-release form to the standard deviation of the peak/trough modulation of the form involved in the use according to the invention.

Preferably, the factor g′ for decrease in the standard deviation of the peak/trough modulation is such that: g′≧1.1; preferably, g′≧1.5, and even more preferably, g′ is between 2.5 and 20.

This drug form for modified release of APIa is also designed in such a way that the microcapsules, whilst ingested, are dispersed and individualized when they reach the stomach, which guarantees regular and gradual gastric emptying of the microunits, in the fed state just as in the fasting state, and therefore, ultimately release of the APIa within its gastrointestinal window of bioabsorption.

For the purpose of the invention, the term “dose” denotes the amount of APIa contained in the drug form administered orally;

The term “immediate release” denotes, in the present disclosure, the release, by an immediate-release form (IRF), of most of the amount of APIa in a relatively brief period of time, for example:
at least 70% of the APIa is released in vivo in one hour, preferably in thirty minutes, after oral ingestion;
or at least 70% of the APIa is released in one hour, preferably in thirty minutes, at any pH between 1.4 and 6.8 in an in vitro dissolution test.

All the dissolution profiles to which reference is made in the present disclosure are realized according to the indications of the European Pharmacopoeia, 4^{th} edition, entitled: “Dissolution test for solid oral forms”: type II dissolutest carried out under SINK conditions at 37° C. and with stirring at 100 rpm.

In the present disclosure, the term “modified release” denotes the release of APIa by an oral pharmaceutical formulation, occurring in vivo at a rate less than that of a reference “immediate-release formulation”, IRF*. Such a modified-release formulation can, for example, comprise an immediate-release phase and a slow-release phase. Modified-release formulations are well known this field; see, for example, Remington: The science and practice of pharmacy, 19^{th} edition, Mack publishing Co. Pennsylvania, USA. The modified release can in particular be a sustained and/or controlled, or even delayed, release.

The pharmacokinetic parameters to which reference is made in the present invention are defined in the following way. After oral administration of the pharmaceutical form to a sample of N human individuals, the individual plasma concentration profile is measured in each of the patients, from which the individual pharmacokinetic parameters are drawn: Tmax, Cmax, C18h:
Tmax is the amount of time after which the plasma concentration reaches its maximum, Cmax.
C18h is the plasma concentration 18 hours after administration.

Based on these individual parameters, those skilled in the art conventionally calculate the mean values of these parameters and their standard deviations. Further details on the discussion of these parameters will be found in the work: Pharmacokinetics and pharmacodynamic Data Analysis 3^{rd} ed., J. Gabrelsson et al., Kristianstads Bocktryckeri A B, Sweden, 2000.

The comparison of the parameters C18h and C18h*, and Cmaxand Cmax* is carried out in a statistically significant manner, under the same conditions and at the same dose of APIa.

The peak/trough modulation of the plasma concentration profiles is defined by the mean of the Cmax/C18h ratio for the APIa.

The expression “dispersed and individualized” means that the APIa-based microcapsules are not trapped in a matrix when they reach the stomach just after they have been ingested. The microcapsules become disseminated in the stomach after they have entered the latter.

Advantageously, the drug form according to the invention comprises microgranules for immediate release of APIa.

The secondary advantages of the invention are in particular as follows:
this oral pharmaceutical form of APIa, which can be administered once or twice a day, is such that, once ingested, the APIa that it contains is released in the gastrointestinal tract and bioabsorbed within its absorption window, even if the latter is narrow.
this oral pharmaceutical form of APIa, which can be administered once or twice a day, guarantees that, once the oral pharmaceutical form has been ingested, the APIa that it contains will not pass in front of its bioabsorption window (!!!) without being released.
this oral pharmaceutical form of APIa, which can be administered once or twice a day, guarantees that, once the oral pharmaceutical form has been ingested, the APIa that it contains will be released independently of the open or closed state of the pylorus.
this oral pharmaceutical form of APIa, which can be administered once or twice a day, is not subject, or barely subject, to the phenomenon of inter- or intraindividual variability of gastric emptying and, ultimately, of in vivo absorption of APIa.
this oral pharmaceutical form of APIa, which can be administered once or twice a day, is at least as effective as the immediate-release once-a-day forms currently in use.
this oral pharmaceutical form of APIa, which can be administered once or twice a day and which comprises microcapsules for modified release of APIa, draws some of its advantages from the small size (≦1000 μm) of these microcapsules and the large number thereof (e.g. at least a thousand or so per dose), which allows gradual and well-controlled gastric emptying.
this oral pharmaceutical form of APIa, which can be administered once or twice a day, makes it possible to increase the Tmax of the APIas and also the period of time for which the plasma concentration of APIa is greater than the minimum plasma concentration of APIa, below which the APIa is therapeutically ineffective.
this oral pharmaceutical form of APIa has an in vitro dissolution profile independent of the dose of APIa.
this oral pharmaceutical form of APIa is composed of microparticles which have the same composition by weight irrespective of the doses of APIa.
this oral pharmaceutical form of APIa, which can be administered once or twice a day, is suitable for patients who have difficulty in swallowing, in particular for children or infants who not only cannot swallow, but who, in addition, require and adjustment of the dose administered according to their weight.
this oral pharmaceutical form of APIa, which can be administered once or twice a day, offers the possibility of mixing the APIa with one or more other active ingredients in the same oral form, it being possible for the respective release times of these various active ingredients to be readily adjusted, independently of one another.
this oral pharmaceutical form of APIa can exist in various gallenic presentation forms, including in particular: tablet, sachet, oral suspension, gel capsule, etc.
the oral gallenic form according to the invention consists of a large number (for example of the order of about one to several thousand) of microcapsules (or microgranules for immediate release of APIa, or of a mixture of several types of microcapsules or microgranules), this multiplicity ensuring, statistically, good reproducibility of the kinetics of transit of the APIa throughout the gastrointestinal tract, and, subsequently, good control of bioavailability and better effectiveness.
the use of a mixture of microcapsules with different modified release profiles makes it possible to produce release profiles which ensure, by means of suitable regulation of the various fractions, a constant level of plasma concentration of APIa.
there is less sensitivity to the variability in gastric emptying because the emptying, which takes place over a large number of particles, is statistically more reproducible.
the bringing of the tissues into contact with a high dose of APIa (dose dumping) is prevented. Each microcapsule in fact contains only a very small dose of APIa. The risk of tissue deterioration due to a local overconcentration of APIa is thus done away with.
this pharmaceutical form does not induce any degradation of the starting APIa and preserves the initial polymorphism of the APIa.
their size of less than or equal to 1000 μm and also the characteristics of their possible coating allows the microcapsules to increase their transit time in the upper parts of the gastrointestinal tract, which ensures an increase in the amount of time taken for the APIa to pass in front of its absorption window and thus maximizes the bioavailability of the APIa.

In accordance with the first embodiment of the invention, the drug form is characterized in that 70% of the APIa is released between 1 and 24 h, preferably 2 and 15 h, and more preferably 2 and 12 h.

Advantageously, this drug form is characterized by an in vitro dissolution profile of the oral pharmaceutical form such that, for any value of the time t of between 2 h and t(70%), preferably for any value of time t of between 1 h and t(70%), the percentage of APIa dissolved is greater than or equal to 35 t/t(70%).

The composition of the coating of the microcapsules according to the first embodiment corresponds, advantageously, to one of the following two families A and B:
family A
   1A—at least one film-forming polymer (P1) which is insoluble in the fluids of the tract, present in a proportion of 50% to 90%, preferably 50% to 80% by weight on a dry basis relative to the total mass of the coating composition, and comprising at least one water-insoluble derivative of cellulose;
   2A—at least one nitrogenous polymer (P2) present in a proportion of 2% to 25%, preferably 5% to 15% by weight on a dry basis relative to the total mass of the coating composition and consisting of at least one polyacrylamide and/or one poly-N-vinylamide and/or one poly-N-vinyllactam;
   3A—at least one plasticizer present in a proportion of 2% to 20%, preferably of 4% to 15% by weight on a dry basis relative to the total mass of the coating composition and consisting of at least one of the following compounds: glyceryl esters, phthalates, citrates, sebacates, cetyl alcohol esters, castor oil;
   4A—at least one surfactant and/or lubricant, present in a proportion of 2% to 20%, preferably of 4% to 15% by weight on a dry basis relative to the total mass of the coating composition and chosen from anionic surfactants and/or from nonionic surfactants and/or from lubricants; it being possible for said surfactant and/or lubricant to comprise just one or a mixture of the abovementioned products;
family B
   1B—at least one film-forming polymer which is insoluble in the fluids of the gastrointestinal tract,
   2B—at least one water-soluble polymer,
   3B—at least one plasticizer,
   4B—and, optionally, at least one surfactant/lubricant, preferably consisting of at least one anionic surfactant and/or at least one nonionic surfactant.

According to a preferred mode of the invention, the families A and B from which the constituents of the coating composition are chosen are as follows:
family A
   1A—ethylcellulose and/or cellulose acetate;
   2A—polyacrylamide and/or polyvinylpyrrolidone;
   3A—castor oil;
   4A—alkali metal or alkaline earth metal salt of fatty acids, stearic acid and/or oleic acid being preferred, a polyoxyethylenated sorbitan ester, derivatives of polyoxyethylenated castor oil, a stearate, preferably calcium stearate, magnesium stearate, aluminum stearate or zinc stearate, a stearyl fumarate, preferably sodium stearyl fumarate, glyceryl behenate; taken on their own or as a mixture with one another;
family B:
   1B
      water-insoluble derivatives of cellulose, ethylcellulose and/or cellulose acetate being particularly preferred,
      water-insoluble acrylic polymers,
      polyvinyl acetates,
      and mixtures thereof;
   2B
      water-soluble derivatives of cellulose,
      polyacrylamides,
      poly-N-vinylamides,
      poly-N-vinyllactams,
      polyvinyl alcohols (PVAs),
      polyoxyethylenes (POEs),
      polyvinylpyrrolidones (PVPs) (the latter being preferred),
      and mixtures thereof;
   3B
      glycerol and its esters, preferably from the following subgroup: acetylated glycerides, glyceryl monostearate, glyceryl triacetate, glyceryl tributyrate,
      phthalates, preferably from the following subgroup: dibutyl phthalate, diethyl phthalate, dimethyl phthalate, dioctyl phthalate,
      citrates, preferably from the following subgroup: acetyl tributyl citrate, acetyl triethyl citrate, tributyl citrate, triethyl citrate,
      sebacates, preferably from the following subgroup: diethyl sebacate, dibutyl sebacate,
      adipates,
      azelates,
      benzoates,
      plant oils,
      fumarates, preferably diethyl fumarate,
      malates, preferably diethyl malate,
      oxalates, preferably diethyl oxalate,
      succinates, preferably dibutyl succinate,
      butyrates,
      cetyl alcohol esters,
      salicylic acid,
      triacetin,
      malonates, preferably diethyl malonate,
      castor oil (the latter being particularly preferred),
      and mixtures thereof;
   4B
      alkali metal or alkaline earth metal salts of fatty acids, stearic acid and/or oleic acid being preferred,
      polyoxyethylenated oils, preferably polyoxy-ethylenated hydrogenated castor oil,
      polyoxyethylene-polyoxypropylene copolymers,
      polyoxyethylenated sorbitan esters,
      polyoxyethylenated castor oil derivatives,
      stearates, preferably calcium stearate, magnesium stearate, aluminum stearate or zinc stearate,
      stearyl fumarates, preferably sodium stearyl fumarate,
      glyceryl behenate,
      and mixtures thereof.

Preferably, the film coating consists of a single layer, the mass of which represents from 1% to 50% by weight, preferably from 5% to 40% by weight, of the total mass of the microcapsules.

Other details and examples of compositions and of methods for obtaining the microcapsules according to the first embodiment according to the invention are given in WO-A-03/084518, the content of which is integrated into the present disclosure by way of reference. For further details in qualitative and quantitative terms, as regards the coating composition of family A, reference will be made to European patent EP-B-0 709 087, the content of which is integrated into the present disclosure by way of reference.

In accordance with a second embodiment of the invention, the oral drug form is such that:
the release of APIa is controlled by two distinct triggering mechanisms, one being based on a variation in pH and the other allowing the release of APIa after a predetermined residence time in the stomach;
at constant pH 1.4, the dissolution profile contains a lag phase which lasts less than or equal to 7 hours, preferably less than or equal to 5 hours, and even more preferably between 1 and 5 hours,
and the passing from pH 1.4 to pH 7.0 results in a release phase which begins without any lag time.

In accordance with the second embodiment of the invention, the pharmaceutical form has an in vitro dissolution profile which may be as indicated below:
at least 20% of the APIa is released after 2 hours at pH=1.4;
at least 50% of the APIa is released after 16 hours at pH=1.4.

Advantageously, the microcapsules for modified release of APIa, according to the second embodiment of the invention, have the following specificities:
the coating allowing the modified release of APIa comprises a composite material
   containing:
      at least one hydrophilic polymer I bearing groups which are ionized at neutral pH,
      at least one hydrophobic compound II;
   representing a mass fraction (% by weight relative to the total mass of the microcapsules) ≦40; and
their mean diameter is less than 2000 μm, and preferably between 50 and 800 μm, and even more preferably between 100 and 600 μm.

According to another advantageous characteristic, the composite material I-II of the coating allowing the modified release of APIa is such that:
the weight ratio II/I is between 0.2 and 1.5, preferably between 0.5 and 1.0,
and the hydrophobic compound II is selected from products which are crystalline in the solid state and which have a melting point TmII≧40° C., preferably TmII≧50° C., and even more preferably 40° C.≦TmII≦90° C.

According to an embodiment of predilection, the hydrophilic polymer I is chosen from:
I.a copolymers of (meth)acrylic acid and of an alkyl ester of (meth)acrylic acid, and mixtures thereof;
I.b cellulose derivatives, preferably cellulose acetates, cellulose phthalates, cellulose succinates, and mixtures thereof, and even more preferably hydroxypropylmethylcellulose phthalates, hydroxylpropylmethylcellulose acetates, hydroxypropylmethylcellulose succinates, and mixtures thereof;
and mixtures thereof.

The polymers I which are even more preferred are copolymers of (meth)acrylic acid and of alkyl (e.g. C₁-C₆ alkyl) esters of (meth)acrylic acid. These copolymers are, for example, of the type such as those sold by the company Röhm Pharma Polymers under the registered trade marks Eudragit®, of the L and S series (such as, for example, Eudragit® L100, S100, L30 D-55 and L100-55). These copolymers are anionic enteric copolymers which are soluble in an aqueous medium at pHs greater than those encountered in the stomach.

Still according to the embodiment of the predilection, the compound II is chosen from the group of following products:
II.a—plant waxes taken on their own or as mixtures with one another;
II.b—hydrogenated plant oils taken on their own or as mixtures with one another;
II.c—mono- and/or di- and/or trimesters of glycerol and of at least one fatty acid;
II.d—mixtures of monoesters, of diesters and of triesters of glycerol and of at least one fatty acid;
II.e—and mixtures thereof.

Even more preferably, the compound II is chosen from the group of following products: hydrogenated cottonseed oil, hydrogenated soybean oil, hydrogenated palm oil, glyceryl behenate, hydrogenated castor oil, tristearin, tripalmitin, trimyristin, yellow wax, hard fat or fat useful as suppository bases, anhydrous dairy fats, lanolin, glyceryl palmitostearate, glyceryl stearate, lauryl macrogol glycerides, cetyl alcohol, polyglyceryl diisostearate, diethylene glycol monostearate, ethylene glycol monostearate, omega-3, and any mixture with one another, preferably from the subgroup of following products: hydrogenated cottonseed oil, hydrogenated soybean oil, hydrogenated palm oil, glyceryl behenate, hydrogenated castor oil, tristearin, tripalmitin, trimyristin, and any mixture with one another.

In practice, and without this being limiting, the compound II is preferably chosen:
from the group of products sold under the following trade marks: Dynasan®, Cutina®, Hydrobase®, Dub®, Castorwax®, Croduret®, Compritol®, Sterotex®, Lubritab®, Apifil®, Akofine®, Softtisan®, Hydrocote®, Livopol®, Super Hartolan®, MGLA®, Corona®, Protalan®, Akosoft®, Akosol®, Cremao®, Massupol®, Novata®, Suppocire®, Wecobee®, Witepsol®, Lanolin®, Incromega®, Estaram®, Suppoweiss®, Gelucire®, Precirol®, Emulcire®, Plurol Diisostearique®, Geleol®, Hydrine®, Monthyle®, and mixtures thereof;
and also from the group of additives for which the codes are as follows: E 901, E 907, E 903, and mixtures thereof;
and, preferably, from the group of products sold under the following trademarks: Dynasan® P60, Dynasan® 114, Dynasan® 116, Dynasan® 118, Cutina® HR, Hydrobase® 66-68, Dub® HPH, Compritol® 888, Sterotex® NF, Sterotex® K, Lubritab®, and mixtures thereof.

According to another advantageous characteristic of the invention, the coating allowing the modified release of APIa is free of talc.

Advantageously, the coating of the microcapsules can comprise, in addition to the essential constituents I and II, other conventional ingredients known to those skilled in the art, such as, in particular:
colorants,
plasticizers, such as, for example, dibutyl sebacate,
hydrophilic compounds, such as, for example, cellulose and its derivatives or polyvinylpyrrolidone and its derivatives,
and mixtures thereof.

Without it being limiting and according to an even more preferred embodiment, the coating of the microcapsules for modified release of APIa comprises a single composite I-II film coating.

Other details and examples of compositions and of methods for obtaining the microcapsules according to the second embodiment according to the invention are given in WO-A-03/030878, the content of which is integrated into the present disclosure by way of reference.

In quantitative terms, the monolayer of coating can represent, for example, at most 40%, preferably at most 30% by weight of the microcapsules. Such a limited degree of coating makes it possible to produce gallenic units which each contain a high dose of active ingredient, without exceeding a size which is completely unacceptable with regard to swallowing. The observance and therefore the success of the treatment can only be improved by this.

According to a third embodiment of the invention, the oral pharmaceutical form according to the invention comprises at least two populations of microcapsules for modified release of APIa. Each population of microcapsules for modified release of APIa can be in accordance with the first or with the second embodiment of the invention.

According to a variant—2i—of the second embodiment of the invention combined with the third embodiment, the oral pharmaceutical form according to the invention comprises at least two populations of microcapsules having different dissolution profiles, for at least one pH value of between 1.4 and 7.4.

According to a variant—2ii—of the second embodiment of the invention combined with the third embodiment, the oral pharmaceutical form according to the invention comprises at least two populations of microcapsules for modified release of APIa which differ by virtue of their respective triggering pHs.

According to yet another variant—2iii—of the second embodiment of the invention combined with the third embodiment, the oral pharmaceutical form according to the invention comprises at least two populations of microcapsules for modified release of APIa which differ by virtue of their respective triggering times.

According to a fourth embodiment of the invention, the oral pharmaceutical form according to the invention comprises at least one population of microcapsules for modified release of APIa and at least one population of microgranules for immediate release of APIa.

According to a variant—2iv—of the second embodiment of the invention combined with the fourth embodiment, the oral pharmaceutical form according to the invention comprises:
at least one population of microgranules for immediate release of APIa;
at least one population P1 of microcapsules for modified release of APIa, and
at least one population P2 of microcapsules for modified release of APIa;
and, moreover, the respective triggering pHs of P1 and of P2 differ by at least 0.5 pH unit, preferably by at least 0.8 pH unit, and even more preferably by at least 0.9 pH unit.

Advantageously, the respective triggering pHs of the various populations of microcapsules for modified release of APIa are between 5 and 7.

According to a variant—2v—of the second embodiment of the invention combined with the fourth embodiment, the oral pharmaceutical form according to the invention comprises:
at least one population of microgranules for immediate release of APIa;
at least one population P1′ of microcapsules for modified release of APIa, the triggering pH of which is equal to 5.5; and
at least one population P2′ of microcapsules for modified release of APIa, the triggering pH of which is between 6.0 inclusive and 6.5 inclusive.

The populations P1, P2, P1′ and P2′ of the variants—2iv—and —2v—of the 2^{nd} embodiment comprise microcapsules for modified release of APIa, obtained in accordance with the 2^{nd} embodiment of the invention.

To illustrate the variants according to which microgranules for immediate release of APIa are present in the pharmaceutical form according to the invention, it can be specified that these variants can correspond to cases where this pharmaceutical form comprises, for example, at least one population of microgranules for immediate release of APIa, the behavior of which in an in vitro dissolution test is such that at least 80% of APIa is released in 1 hour at any pH between 1.4 and 7.4.

The drug form according to the invention can comprise, in addition to the microunits consisting of the microcapsules for modified release of APIa, microunits of APIa other than microcapsules, i.e. microgranules for immediate release of APIa and/or of (an) other active ingredient(s) API(s). These immediate-release microgranules are advantageously uncoated and can be of the same type as those used in the preparation of the microcapsules according to the invention.

In addition, all the microunits (microcapsules and, optionally, microgranules) constituting the medicament according to the invention can be formed by various populations of microunits, these populations differing from one another at least by virtue of the nature of the active ingredient(s) other than the APIa contained in these microunits and/or by virtue of the amount of APIa or of other optional active ingredient(s) that they contain and/or by virtue of the composition of the coating and/or by virtue of the fact that they are modified-release or immediate release.

According to a specific embodiment, the drug form according to the invention is in the form of single oral daily dose comprising from 1000 to 500 000 microunits containing APIa.

According to another specific embodiment, the drug form according to the invention is in the form of a single oral daily dose comprising from 1000 to 500 000 microcapsules for modified release of APIa.

According to a variant, the drug form according to the invention comprises at least one suspension of microcapsules of APIa in an aqueous liquid phase which is preferably saturated or which becomes saturated with APIa on contact with the microcapsules, the coating of said microcapsules preferably having a composition corresponding to one of the following two families A′ and B′:
family A′
   1A′—at least one film-forming polymer (P1) which is insoluble in the fluids of the tract, present in a proportion of 50% to 90%, preferably 50% to 80% by weight on a dry basis relative to the total mass of the coating composition, and comprising at least one water-insoluble derivative of cellulose;
   2A′—at least one nitrogenous polymer (P2) present in a proportion of 2% to 25%, preferably 5% to 15% by weight on a dry basis relative to the total mass of the coating composition and consisting of at least one polyacrylamide and/or one poly-N-vinylamide and/or one poly-N-vinyllactam;
   3A′—at least one plasticizer present in a proportion of 2% to 20%, preferably of 4% to 15% by weight on a dry basis relative to the total mass of the coating composition and consisting of at least one of the following compounds: glyceryl esters, phthalates, citrates, sebacates, cetyl alcohol esters, castor oil;
   4A′—at least one surfactant and/or lubricant, present in a proportion of 2% to 20%, preferably of 4% to 15% by weight on a dry basis relative to the total mass of the coating composition and chosen from anionic surfactants and/or from nonionic surfactants and/or from lubricants; it being possible for said surfactant and/or lubricant to comprise just one or a mixture of the abovementioned products;
family B′
   1B′—at least one film-forming polymer which is insoluble in the fluids of the gastrointestinal tract,
   2B′—at least one water-soluble polymer,
   3B′—at least one plasticizer,
   4B′—and, optionally, at least one surfactant/lubricant, preferably selected from the group of following products:
      anionic surfactants,
      and/or nonionic surfactants.

In practice, the coating composition families A′ and B′ are, for example, as follows:
family A′:
   1A′—ethylcellulose and/or cellulose acetate;
   2A′—polyacrylamide and/or polyvinylpyrrolidone;
   3A′—castor oil;
   4A′—alkali metal or alkaline earth metal salt of fatty acids, stearic acid and/or oleic acid being preferred, polyoxyethylenated sorbitan esters, derivatives of polyoxyethylenated castor oil, stearates, preferably calcium stearate, magnesium stearate, aluminum stearate or zinc stearate, stearyl fumarate, preferably sodium stearyl fumarate, glyceryl behenate; taken alone or as a mixture with one another;
family B′:
   1B′
      water-insoluble derivatives of cellulose, ethylcellulose and/or cellulose acetate being particularly preferred,
      acrylic polymers,
      polyvinyl acetates,
      and mixtures thereof;
   2B′
      water-soluble derivatives of cellulose,
      polyacrylamides,
      poly-N-vinylamides,
      poly-N-vinyllactams,
      polyvinyl alcohols (PVAs),
      polyoxyethylenes (POEs),
      polyvinylpyrrolidones (PVPs) (the latter being preferred),
      and mixtures thereof;
   3B′
      glycerol and its esters, preferably from the following subgroup: acetylated glycerides, glyceryl monostearate, glyceryl triacetate, glyceryl tributyrate,
      phthalates, preferably from the following subgroup: dibutyl phthalate, diethyl phthalate, dimethyl phthalate, dioctyl phthalate,
      citrates, preferably from the following subgroup: acetyl tributyl citrate, acetyl triethyl citrate, tributyl citrate, triethyl citrate,
      sebacates, preferably from the following subgroup: diethyl sebacate, dibutyl sebacate,
      adipates,
      azelates,
      benzoates,
      plant oils,
      fumarates, preferably diethyl fumarate,
      malates, preferably diethyl malate,
      oxalates, preferably diethyl oxalate,
      succinates, preferably dibutyl succinate,
      butyrates,
      cetyl alcohol esters,
      salicylic acid,
      triacetin,
      malonates, preferably diethyl malonate,
      castor oil (the latter being particularly preferred),
      and mixtures thereof;
4B′
   alkali metal or alkaline earth metal salts of fatty acids, stearic acid and/or oleic acid being preferred,
   polyoxyethylenated oils, preferably polyoxy-ethylenated hydrogenated castor oil,
   polyoxyethylene-polyoxypropylene copolymers,
   polyoxyethylenated sorbitan esters,
   polyoxyethylenated castor oil derivatives,
   stearates, preferably calcium stearate, magnesium stearate, aluminum stearate or zinc stearate,
   stearyl fumarates, preferably sodium stearyl fumarate,
   glyceryl behenate,
   and mixtures thereof.

According to an advantageous mode of this variant, in which the drug form is in suspension, it is envisaged that this suspension comprises means (b) containing “viscosity-modifying” excipients, which are in the form of coated particles, each coated with at least one hydrophobic film-coating.

This hydrophobic film-coating comprises, for example, at least one product chosen from the group comprising polymers which are insoluble in the fluids of the tract.

This variant makes it possible, during a normal use, for these viscosity-modifying agents to remain encapsulated and therefore inactive. In the event of misuse involving crushing, the hydrophobic film-coating of these viscosity-modifying agents cracks, and the latter are then released and can perform their function, leading to a significant increase in viscosity, putting a stop to any misuse by injection.

Advantageously, the coating for coating (controlling the diffusion of the APIa) of the microcapsules of the suspension consists of a single layer.

This suspension contains, e.g:
30% to 90% by weight, preferably 60% to 85% by weight of liquid phase (advantageously of aqueous solution),
5% to 70% by weight, preferably 15% to 40% by weight of microcapsules.

In practice, the amount of APIa solvent liquid phase (preferably aqueous solution) is preferably such that the proportion of APIa dissolved and originating from the microcapsules is less than or equal to 15%, preferably less than or equal to 5% by weight relative to the total mass of APIa contained in the microcapsules.

Preferably, the liquid phase is at least partly, preferably completely, saturated with APIa subsequent to the incorporation of the microcapsules into this liquid phase.

An alternative for this suspension is for the saturation with APIa to take place by means of the APIa contained in the microcapsules.

Another alternative for this suspension is for the liquid phase to be at least partly, preferably completely, saturated with APIa by means of nonencapsulated APIa. Another alternative for this suspension is that it is in the form of a powder for oral suspension to be reconstituted: the powder contains all the elements of the suspension described above, except the water (or the liquid phase), which is added by the user.

Besides the liquid forms, the drug form according to the invention can be in the form of a sachet of microcapsule powder, of a tablet obtained from microcapsules, or of a gel capsule containing microcapsules.

According to another of its aspects, the invention also encompasses the use of the microcapsules for modified release of APIa as defined above, and optionally of the microgranules for immediate release of APIa as defined above, for the preparation of pharmaceutical, microparticulate oral galenic forms, preferably in the form of tablets, advantageously orodispersible tablets, of powders, of gel capsules or of suspensions.

According to yet another of its aspects, the invention also encompasses the use of the microcapsules for modified release of APIa as defined above, and optionally of the microgranules for immediate release of APIa as defined above, for the preparation of a therapeutically safe, microparticulate oral pharmaceutical form designed in such a way that, once said pharmaceutical form has been ingested, the microcapsules that it contains are dispersed and individualized when they reach the stomach, which allows these microcapsules to be subjected to regular and gradual gastric emptying, whether the patient had eaten or was fasting at the time the dose was taken, thus guaranteeing a release of APIa within its window of bioabsorption.

The APIa used belongs, for example, to at least one of the following families of active substances: amphetamines, analgesics, anorexigens, antalgics, antidepressants, antiepileptics, antimigraine agents, antiparkinsonian agents, antitussives, anxiolytics, barbiturates, benzodiazepines, hypnotics, laxatives, neuroleptics, opiates, psychostimulants, psychotropic agents, sedatives and stimulants.

Even more specifically, the APIa used is chosen from the following compounds: acetorphine, acetylalphamethylfentanyl, acetyldihydrocodeine, acetylmethadol, alfentanil, allylprodine, alphacetylmethadol, alphameprodine, alphamethadol, alphamethylfentanyl, alpha-methylthofentanyl, alphaprodine, anileridine, atropine, benzethidine, benzylmorphine, beta-hydroxyfentanyl, beta-hydroxymethyl-3-fentanyl, beta-cetylmethadol, betameprodine, betamethadol, betaprodine, bezitramide, buprenorphine, dioxaphetyl butyrate, cannabis, cetobemidone, clonitazene, codeine, coca, cocaine, codoxime, concentrate of poppy straw, desomorphine, dextromoramide, dextropropoxyphene, diampromide, diethylthiambutene, difenoxine, dihydrocodeine, dihydroetorphine, dihydromorphine, dimenoxadol, dimepheptanol, dimethylthiambutene, diphenoxylate, dipipanone, drotebanol, ecgonin, ephedrine, ethylmethylthiambutene, ethylmorphine, etonitazene, etorphine, etoxeridine, fentanyl, furethidine, heroin, hydrocodone, hydromorphinol, hydromorphon, hydroxypethidine, isomethadone, levomethorphan, levomoramide, levophenacylmorphan, levorphanol, meperidine, metazocine, methadone, methyldesorphine, methyldihydromorphine, methylphenidate, methyl-3-thiofentanyl, methyl-3-fentanyl, metopon, moramide, morpheridine, morphine, MPPP, myrophine, nicocodine, nicodicodine, nicomorphine, noracymethadol, norcodeine, norlevorphanol, normethadone, normorphine, norpipanone, opium, oxycodone, oxymorphone, para-fluorofentanyl, PEPAP, pentazocine, pethidine, phenampromide, phenazocine, phenomorphan, phenoperidine, pholcodine, piminodine, piritramide, proheptazine, propanolol, properidine, propiram, racemethorphan, racemoramide, racemorphan, remifentanil, sufentanil, thebacone, thebaine, thiofentanyl, tilidine, trimeperidine, and mixtures thereof.

Advantageously, the APIa is selected from opiates, and more particularly from the group comprising the following compounds:

anileridine, acetorphine, acetylalphamethylfentanyl, acetyldihydrocodeine, acetylmethadol, alfentanil, allylprodine, alphacetylmethadol, alphameprodine, alphaprodine, alphamethadol, alphamethylfentanyl, alpha-methylthiofentanyl, alphaprodine, anileridine, atropine, butorphanol, benzethidine, benzylmorphine, beta-hydroxyfentanyl, beta-hydroxymethyl-3-fentanyl, betacetylmethadol, betameprodine, betamethadol, betaprodine, bezitramide, buprenorphine, dioxaphetyl butyrate, clonitazene, cyclazocine, cannabis, cetobemidone, clonitazene, codeine, coca, cocaine, codoxime, concentrate of poppy straw, dezocine, dimenoxadol, dioxaphetylbutyrate, dipipanone, desomorphine, dextromoramide, dextropropoxyphene, diampromide, diethylthiambutene, difenoxine, dihydrocodeine, dihydroetorphine, dihydromorphine, dimenoxadol, dimepheptanol, dimethylthiambutene, diphenoxylate, dipipanone, drotebanol, eptazocine, ethoheptazine, ethylmethylthiambutene, ethylmorphine, etonitazene, ecgonin, ephedrine, ethylmethylthiambutene, ethylmorphine, etonitazene, etorphine, etoxeridine, fentanyl, furethidine, heroin, hydrocodone, hydromorphinol, hydromorphone, hydroxypethidine, isomethadone, ketobemidone, levallorphan, iofentanil, levomethorphan, levomoramide, levophenacylmorphan, levorphanol, meptazinol, meperidine, metazocine, methadone, methyldesorphine, methyldihydromorphine, methylphenidate, methyl-3-thiofentanyl, methyl-3-fentanyl, metopon, moramide, morpheridine, morphine, MPPP, myrophine, nalbuphine, narceine, nicomorphine, norlevorphanol, normethadone, nalorphine, normorphine, nicocodine, nicodicodine, nicomorphine, noracymethadol, norcodeine, norlevorphanol, normethadone, normorphine, norpipanone, opium, oxycodone, oxymorphone, papavereturn, phenadoxone, phenoperidine, promedol, properidine, propiram, propoxyphene, para-fluorofentanyl, PEPAP, pentazocine, pethidine, phenampromide, phenazocine, phenomorphan, phenoperidine, pholcodine, piminodine, piritramide, proheptazine, propanolol, properidine, propiram, racemethorphan, racemoramide, racemorphan, remifentanil, sufentanil, thebacone, thebaine, thiofentanyl, tilidine, trimeperidine, tramodol, pharmaceutically acceptable salts of these compounds and mixtures of these compounds and/or of their salts.

The drug forms according to the invention can comprise at least one other active ingredient other than an APIa. The abbreviation API will hereinafter denote, without distinction, one or more active ingredients other than an APIa.

The in vivo or in vitro release of the API can be immediate or modified. The API can be contained in microgranules for immediate release of the API or in microcapsules for modified release of the API.

This API can be chosen, inter alia, from the group comprising antidepressants, amphetamines, anorexigens, analgesics, antiepileptics, antimigraine agents, antiparkinsonian agents, antitussives, anxiolytics, barbiturates, benzodiazepines, hypnotics, laxatives, neuroleptics, psychostimulants, psychotropic agents, sedatives, stimulants, anti-inflammatories, pharmaceutically acceptable salts of these compounds and mixtures of these compounds and/or of their salts.

By way of examples of anti-inflammatories, mention may, for example, by made of ibuprofen, acetaminophen, diclofenac, naproxene, benoxaprofen, flurbiprofen, fenoprofen, flubufen, ketoprofen, indoprofen, piroprofen, carprofen, oxaprozine, pramoprofen, muroprofen, trioxaprofen, suprofen, aminoprofen, tiaprofenic acid, fluprofen, bucloxic acid, indomethacin, sulindac, tolmetine, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam or isoxicam, pharmaceutically acceptable salts of these compounds and mixtures of these compounds and/or of their salts.

In accordance with an advantageous mode of the invention, the drug form comprises at least two populations of microcapsules having different release profiles according to the similarity factor f2.

The nonlimiting examples which follow make it possible to understand the invention more clearly and to reveal its advantages.

### DESCRIPTION OF THE FIGURES

FIG. 1 represents the dissolution profile in a reference test (% dissolution D as a function of time T), in vitro, of the microparticles without protective overcoating of Example 1.

FIG. 2 represents the dissolution profile in the reference test (% dissolution D as a function of time T), in vitro, of the microparticles with protective overcoating of Example 2, before crushing.

FIG. 3 represents the dissolution profile in the reference test (% dissolution D as a function of time T), in vitro, of the microparticles with protective overcoating of Example 2, after crushing.

FIG. 4 represents the profile for increase in viscosity V in mPa·s as a function of time T in hours, of the microparticles of viscosity-modifying agents for Example 3, placed in the presence of water.

FIG. 5 represents the release profiles (% by weight of APIa as a function of time in hours) of an intact tablet and of the crushed tablet and at pH 1.4. Legend: -▪- intact tablet, -□- crushed tablet.

FIG. 6 represents the release profiles (% by weight of APIa as a function of time in hours) of the intact and crushed microcapsules at pH 1.4. Legend: -▴- anti-misuse microcapsules; -Δ- crushed anti-misuse microcapsules.

The reference dissolution test in the examples which follow is an in vitro dissolution test carried out according to the indications of the European Pharmacopoiea, 4^{th} edition, entitled: “Dissolution test for solid oral forms”: type II dissolutest carried out under SINK conditions, maintained at 37° C. and stirred at 100 rpm.

In some of the following examples, metformin is used as model active ingredient. Metformin hydrochloride has a solubility and a stability comparable to oxycodone hydrochloride.

### EXAMPLE 1

### Preparation of Microparticles of Acyclovir (Model Active Ingredient)

### Step 1: Granules

45 g of acyclovir, 25 g of PEG 40-hydrogenated castor oil and 30 g of povidone are solubilized beforehand in a water/acetone/isopropanol mixture (5/57/38 m/m). This solution is then sprayed onto 800 g of cellulose spheres (of diameter between 100 and 200 μm) in a Glatt QPC-G1 fluidized airbed device.

### Step 2: Coating

50 g of granules obtained above are coated with 6.5 g of ethylcellulose, 0.5 g of castor oil, 0.5 g of PEG 40-hydrogenated castor oil (BASF) and 2.5 g of povidone dissolved in an acetone/isopropanol mixture (60/40 m/m), in a miniGlatt fluidized airbed device.

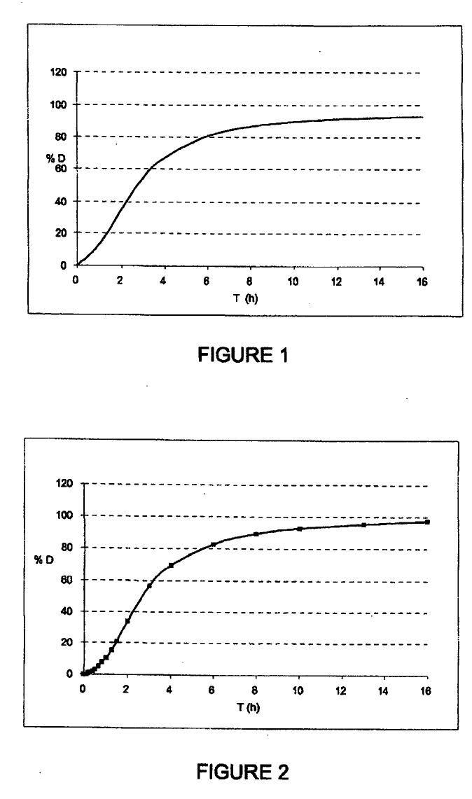

The average diameter of the particles obtained is 180 μm. These microparticles are virtually spherical and release their content over approximately 8 hours in the reference dissolution test (FIG. 1).

### EXAMPLE 2

### Overcoating of the Microparticles of Acyclovir in Such a Way as to Minimize their Sensitivity to Crushing

10 g of ethylcellulose, 25 g of PEG 6000 and 5 g of magnesium stearate are dispersed in 160 g of isopropanol. This dispersion is then sprayed onto 40 g of microparticles obtained at the end of the second step of Example 1.

In this example, the protective layer (or overcoating) does not modify the kinetics of release of API in the reference dissolution test (FIG. 2).

The average diameter of the microparticles obtained is 250 μm.

When these objects are subjected to shear, for example in a mortar, the layer containing the ethylcellulose, the PEG 6000 and the magnesium stearate protects the particle of active ingredient by reducing the shear effects.

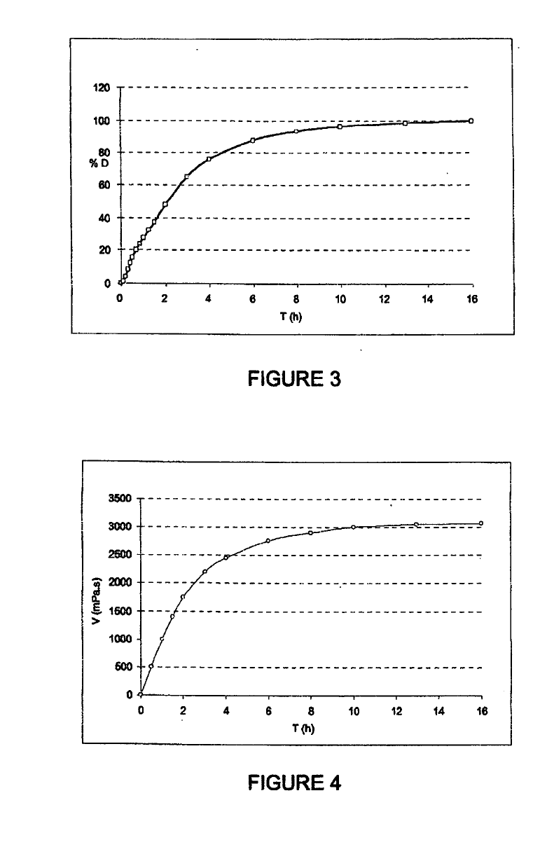

The release kinetics in the reference dissolution test for the microparticles after crushing remain sustained and virtually identical to the starting microparticles (FIG. 3).

### EXAMPLE 3

### Preparation of Microparticles of Viscosity-Modifying Agents

500 g of polyoxyethylene, 80 g of hydroxypropylcellulose and 20 g of ethylcellulose are dispersed in an acetone/isopropanol mixture (60/40 m/m).

This solution is then sprayed onto 400 g of cellulose spheres (of diameter of between 100 and 200 μm) in a Glatt GPC-G1 fluidized airbed device. The average diameter of the microparticles obtained is 260 μm.

2.5 g of microparticles thus obtained are introduced into 100 g of water.

The viscosity at 25° C. over time is given in FIG. 4. At equilibrium, the solution obtained has a viscosity of the order of 3000 mPa·s. A solution this viscous cannot be injected.

The kinetics for increasing viscosity are comparable to the release kinetics of the microparticles of API obtained in Examples 1 and 2.

### EXAMPLE 4

The final pharmaceutical form according to the invention is the combination of the microparticles prepared in Example 2 and in Example 3. These two types of microparticles are physically indiscernible (same size, shape, density, etc.).

These microparticles are protected against improper use since they:
conserve a sustained release of the API even after crushing;
very greatly increase the viscosity of an aqueous solution that has been used to extract the API from the microparticles.

### COUNTER EXAMPLE 1

### Tablets According to the Prior Art

Metformin tablets are prepared according to U.S. Pat. No. 5,656,295, Examples 3-4, column 10, lines 20 to 63, replacing the oxycodon with metformin.

### COUNTER EXAMPLE 2

### Crushing of the Tablets According to the Prior Art

A tablet of Counter Example 1 is placed in a glass mortar and crushed. The crushed tablet is tested in a type II dissolutest in accordance with the Pharmacopoeia, at 37° C. and with stirring at 75 rpm, in the following media: i) solution of HCl at pH 1.4.

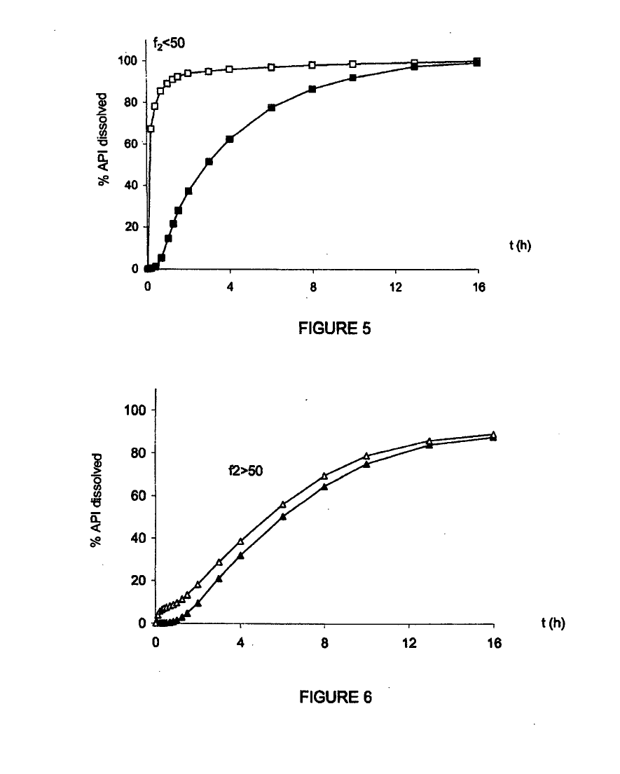

It is noted that the release of the metformin is virtually immediate when the tablet has been crushed beforehand. The dissolution profiles are different according to the similarity factor f2 test: f2<50 (see FIG. 5).

### EXAMPLE 5

### Example According to the Invention

A solution of 755 g of metformin, 55.5 g of PVP and 3889 g of water is film-coated onto 216 g of cellulose neutral carriers. 455 g of metformin granules are film-coated with a mixture of 147 g of ethocel 20P, 7.35 g of PVP, 7.35 g of cremophor RH 40, 34.3 g of castor oil and 2.254 kg of isopropanol. The microcapsules are then dried and screened through 500 μm.

A mixture of 14.2 g of ethocel 20P, 1.5 g of triethyl citrate (TEC), 7.1 g of magnesium stearate, 3.51 g of PEG 6000 and 284 g of ethanol is film-coated onto 55 g of the microcapsules obtained above. The microcapsules are then dried and screened through 500 μm.

### EXAMPLE 6

### Crushing of the Microcapsules According to the Invention

400 mg of microcapsules of Example 5 are placed in a glass mortar and crushed. The microcapsules are recovered and tested in a type II dissolutest in accordance with the Pharmacopoeia, at 37° C. and with stirring at 75 rpm, in a solution of HCl at pH 1.4. FIG. 6 represents the release profiles for the crushed microcapsules and for the intact microcapsules. It is noted that, in this case, the metformin release profile remains sustained and virtually identical to the profile of noncrushed microcapsules of Example 5. The dissolution profiles are similar according to the similarity factor f2 test: f2>50.

### EXAMPLE 7

### Viscosity-Modifying Mixture

The ease with which various viscosity-modifying agents, used alone or as a mixture, can be drawn up is reported in Table 1. The ease with which they can be drawn up was evaluated on insulin syringes having a volume of 1 ml, through a needle (29G, ˜0.33 mm×15 mm). The drawing up was carried out with a sterile cotton filter placed at the end of the needle. The medium is considered to be nonpumpable if the time required to draw up 1 ml is greater than 5 min.

TABLE 1

| Compound/Solvent | Water | Vodka | 99% ethanol |
|---|---|---|---|
| A = Rhodigel | Nonpumpable | Nonpumpable | Pumpable |
| (40 mg/1 ml) | | | (insoluble) |
| B = Ethocel 100P | Pumpable | Pumpable | Nonpumpable |
| (40 mg/1 ml) | (insoluble) | (insoluble) | |
| C = Natrosol 250 | Nonpumpable | Nonpumpable | Pumpable |
| HHX (40 mg/1 ml) | | | (insoluble) |
| Mixture ABC | Nonpumpable | Nonpumpable | Nonpumpable |
| (3 × 40 = 120 | | | |
| mg/1 ml) | | | |

The viscosity-modifying agents taken separately are not soluble and viscous in all the solvents. The mixture of the viscosity-modifying agents makes it possible to achieve viscosities which are sufficient for the system not to be pumpable in the three media considered.

### EXAMPLE 8

Example According to the Invention of Particles of Viscosity-Modifying Agents to be Incorporated into a Sachet or Suspension Formulation for the Purpose of Preventing Misuse by Injection of a Suspension

6 g of PVP, 30 g of Rhodigel, 30 g of Ethocel 100P and 30 g of Natrosol 250 HHX are granulated with a solution of ethanol. 1 g of triethyl citrate is added, with stirring, to a solution of 8 g of Ethocel 07P, 2.1 g of stearyl alcohol and 110 g of ethanol at 70° C. After homogenization, the solution is then sprayed onto 50 g of granules obtained previously.

The rheological behavior after dispersion in water of the film-coated granules in the intact form and after crushing is reported in Table 2:

TABLE 2

| | Uncrushed film-coated | Crushed film-coated |
|---|---|---|
| | granule (50 mg/1 ml) | granule (50 mg/1 ml) |
| Dispersion in | Nonviscous | Viscous, nonpumpable |
| water: | | |

The combination of these particles with the microcapsules of APIa makes it possible:
to correctly treat patients by providing them with a suspension that is easy to swallow,
to combat misuse by means of a drastic increase in viscosity after crushing and suspension.

### EXAMPLE 9

### EXAMPLE, ACCORDING TO THE INVENTION, OF A Combination

A mixture of 65 g of paracetamol, 10 g of talc, 5.5 g of PVP and 350 g of water is film-coated onto 22 g of cellulose neutral carriers. A mixture of 14.2 g of ethocel 20P, 5.1 g of PEG 6000, 1.5 g of triethyl citrate and 284 g of ethanol is film-coated onto 55 g of the microcapsules previously obtained. The microcapsules are then dried and screened through 500 μm.

A gel capsule is filled with the following mixture: 300 mg of the microcapsules of paracetamol previously obtained, 15 mg of microcapsules of Example 5 and 3 mg of magnesium stearate. In the mixture thus formed, the microcapsules of paracetamol and of metformin cannot be discerned by size, by shape or by color.

These microcapsules of paracetamol are immediate-release, IR, capsules. In the event of crushing in the case of an attempt at misuse, these microcapsules of paracetamol offer no resistance to the crushing, whereas the microcapsules of metformin according to the invention are protected by virtue of their overcoating (cf. Example 5 above).

What is claimed is:

1. A solid oral drug form comprising anti-misuse means and at least one active ingredient (API), characterized in that at least a part of the API is contained in microparticles, and characterized in that the anti-misuse means comprise anti-crushing means (a) provided so as to allow the microparticles of API to withstand crushing, so as to prevent misuse.

2. The drug form as claimed in claim 1, characterized in that it comprises means (b) provided for preventing misuse of the API after a possible liquid extraction.

3. The drug form as claimed in any one of the preceding claims, characterized in that the anti-crushing means (a) are:
an overcoating for protection of the microparticles of API having at least one of the following characteristics:
a viscoelastic properties for absorbing the energy dissipated during crushing,
a low cohesive strength for promoting breaking of the overcoating and not of the microparticles,
a low surface energy for promoting slipping of the microparticles during crushing,
an ability to form a paste under high shear,
and/or excipients in the free state, i.e. not contained in nor supported by microparticles, and capable of acting against, or even preventing, the crushing of the microparticles of API.

4. The drug form as claimed in any one of the preceding claims, characterized in that at least a part of the microparticles of API are microparticles for modified release of API, preferably microcapsules for modified release of API.

5. The drug form as claimed in claims 3 and 4, characterized in that the overcoating for the protection of the microparticles for modified release of API is designed in such a way that it makes it possible, in the event of crushing, to maintain a non-immediate release for at least a part of said microparticles for modified release of API.

6. The drug form as claimed in claim 3 or 5, characterized in that the overcoating for the protection of the microparticles of API comprises:
(i) at least one film-forming compound which ensures the cohesion of the overcoating, and at least one of the following three compounds:
(ii) a lubricant/caking agent,
(iii) a viscoelastic compound,
(iv) a plasticizer.

7. The drug form as claimed in claim 6, characterized in that the film-forming compound (i) is chosen from:
cellulose derivatives,
acrylic derivatives,
and mixtures thereof.

8. The drug form as claimed in claim 6, characterized in that the lubricant/caking agent (ii) is chosen from the group comprising:
stearic acid and stearates, preferably calcium stearate, zinc stearate or magnesium stearate;
magnesium oxide;
poloxamers;
sodium benzoate;
anionic, cationic or nonionic surfactants;
starches, preferably corn starch;
talc;
colloidal silica;
waxes, preferably hydrogenated plant oils, and even more preferably hydrogenated cottonseed oils, hydrogenated soybean oils, hydrogenated palm oils, hydrogenated castor oils; glyceryl behenates, tristearins, tripalmitins, trimyristins, yellow waxes, hard fats, anhydrous dairy fats, lanolins, glyceryl palmitostearates, glyceryl stearates, lauric acid macrogol glycerides, cetyl alcohols, glyceryl diisostearates, diethylene glycol monostearates, ethylene monostearates, omegas-3, and mixtures thereof;
fatty bases for suppositories, comprising glycerol, triglycerides, theobroma oils, cocoa butters, and mixtures thereof;
and mixtures thereof.

9. The drug form as claimed in claim 6, characterized in that the viscoelastic agent (iii) is selected from the group of following products:
poly-N-vinylamides,
gum bases,
fatty alcohols,
poly-N-vinyllactams,
polyvinyl alcohols (PVAs),
polyoxyethylenes (POEs),
polyethylene glycols (PEGs),
polydextroses,
hydrogenated mono-, di- and polysaccharides,
polyvinylpyrrolidones (PVPs) (the latter being preferred),
and mixtures thereof.

10. The drug form as claimed in claim 6, characterized in that the plasticizer (iv) is selected from the group of following products:
glycerol and its esters, preferably from the following subgroup: acetylated glycerides, glyceryl monostearate, glyceryl triacetate, glyceryl tributyrate,
phthalates, preferably from the following subgroup: dibutyl phthalate, diethyl phthalate, dimethyl phthalate, dioctyl phthalate,
citrates, preferably from the following subgroup: acetyl tributyl citrate, acetyl triethyl citrate, tributyl citrate, triethyl citrate,
sebacates, preferably from the following subgroup:
diethyl sebacate, dibutyl sebacate,
adipates,
azelates,
benzoates,
plant oils, preferably cottonseed oils, soybean oils, palm oils, castor oils, and mixtures thereof;
fumarates, preferably diethyl fumarate,
malates, preferably diethyl malate,
oxalates, preferably diethyl oxalate,
succinates, preferably dibutyl succinate,
butyrates,
cetyl alcohol esters,
triacetin,
malonates, preferably diethyl malonate,
and mixtures thereof.

11. The drug form as claimed in claim 3, characterized in that the excipients included in the anti-crushing means (a) are chosen from:
compression agents,
and/or inert microbeads,
and/or gum bases,
and/or viscoelastic agents of the type such as those defined above in claim 9.

12. The drug form as claimed in claim 2, characterized in that the means (b) provided for preventing misuse of the API after a possible liquid extraction comprise “viscosity-modifying” excipients capable of increasing the viscosity of the extraction liquid so as to act against the misuse, in particular by injection.

13. The drug form as claimed in claim 12, characterized in that the “viscosity-modifying” excipients capable of increasing the viscosity of the extraction liquid so as to act against the misuse, in particular by injection, are present:
in and/or on microparticles,
and/or in an overcoating of all or part of the microparticles of API,
and/or in the free state, i.e. not contained in nor carried by microparticles.

14. The drug form as claimed in claim 12 or 13, characterized in that the “viscosity-modifying” excipients are capable of increasing the viscosity of the liquid used for the possible extraction according to kinetics similar to the kinetics of extraction of the API contained in the microparticles, so as to trap the extracted API in the viscous medium.

15. The drug form as claimed in any one of claims 12 to 14, characterized in that the excipients included in the means (b) are chosen from the groups of following polymers:
polyacrylic acids and their derivatives, and/or
polyoxyethylenes (POEs), and/or
polyvinyl alcohols (PVAs),
polyvinylpyrrolidones (PVPs), and/or
gelatins, and/or
cellulose derivatives (e.g. hydroxypropylmethylcellulose, methylcellulose, hydroxyethylcellulose, carboxymethylcellulose), and/or
polysaccharides, preferably from the subgroup comprising: sodium alginate, pectins, guars, xanthans, carragheenans, gellans,
and mixtures thereof.

16. The drug form as claimed in claim 13, characterized in that it comprises means (b) containing “viscosity-modifying” excipients, which are in the form of coated particles, each coated with at least one hydrophobic film-coating.

17. The drug form as claimed in any one of the preceding claims, characterized in that it cannot be converted into a dry form which can be administered by nasal aspiration.

18. The drug form as claimed in any one of the preceding claims, characterized in that it cannot be converted into an injectable form.

19. The drug form as claimed in any one of the preceding claims, characterized in that it comprises immediate-release API and/or modified-release API.

20. The drug form as claimed in any one of the preceding claims, characterized in that extraction of the API by chewing and/or crushing is not effective.

21. The drug form as claimed in one of the preceding claims, characterized in that the API used belongs to at least one of the following families of active substances: amphetamines, analgesics, anorexigens, antalgics, antidepressants, antiepileptics, anti-migraine agents, antiparkinsonian agents, antitussives, anxiolytics, barbiturates, benzodiazepines, hypnotics, laxatives, neuroleptics, opiates, psychostimulants, psychotropic agents, sedatives and stimulants.

22. The drug form as claimed in one of the preceding claims, characterized in that the API used is chosen from the following compounds: acetorphine, acetylalphamethylfentanyl, acetyldihydrocodeine, acetylmethadol, alfentanil, allylprodine, alphacetylmethadol, alphameprodine, alphamethadol, alphamethylfentanyl, alpha-methylthofentanyl, alphaprodine, anileridine, atropine, benzethidine, benzylmorphine, beta-hydroxyfentanyl, beta-hydroxymethyl-3-fentanyl, beta-cetylmethadol, betameprodine, betamethadol, betaprodine, bezitramide, buprenorphine, dioxaphetyl butyrate, cannabis, cetobemidone, clonitazene, codeine, coca, cocaine, codoxime, concentrate of poppy straw, desomorphine, dextromoramide, dextropropoxyphene, diampromide, diethylthiambutene, difenoxine, dihydrocodeine, dihydroetorphine, dihydromorphine, dimenoxadol, dimepheptanol, dimethylthiambutene, diphenoxylate, dipipanone, drotebanol, ecgonin, ephedrine, ethylmethylthiambutene, ethylmorphine, etonitazene, etorphine, etoxeridine, fentanyl, furethidine, heroin, hydrocodone, hydromorphinol, hydromorphone, hydroxypethidine, isomethadone, levomethorphan, levomoramide, levophenacylmorphan, levorphanol, meperidine, metazocine, methadone, methyldesorphine, methyldihydromorphine, methylphenidate, methyl-3-thiofentanyl, methyl-3-fentanyl, metopon, moramide, morpheridine, morphine, MPPP, myrophine, nicocodine, nicodicodine, nicomorphine, noracymethadol, norcodeine, norlevorphanol, normethadone, normorphine, norpipanone, opium, oxycodone, oxymorphone, para-fluorofentanyl, PEPAP, pentazocine, pethidine, phenampromide, phenazocine, phenomorphan, phenoperidine, pholcodine, piminodine, piritramide, proheptazine, propanolol, properidine, propiram, racemethorphan, racemoramide, racemorphan, remifentanil, sufentanil, thebacone, thebaine, thiofentanyl, tilidine, trimeperidine, and mixtures thereof.

23. The drug form as claimed in any one of the preceding claims, characterized in that the microparticles of API have an average diameter of less than or equal to 1000 μm, preferably between 50 and 800 microns, and more preferably between 100 and 600 microns.

24. The oral drug form as claimed in any one of the preceding claims, comprising anti-misuse means and a plurality of microcapsules for modified release of at least one analgesic active ingredient (APIa), at least a part of said microcapsules consisting individually of a nucleus comprising at least one APIa and coated with at least one coating for the modified release of the APIa; the average diameter of said microcapsules being less than or equal to 1000 μm, preferably between 50 and 800 μm, more preferably between 50 and 600 μm, and even more preferably between 80 and 400 μm; characterized:
in that it comprises at least 1000 microcapsules per dose;
and in that the amount of APIa and the modified-release coating are such that they allow an administration once or twice a day for analgesic purposes.

25. The drug form as claimed in claim 24, characterized in that it makes it possible to obtain, after taking one dose, a plasma profile defined as follows:
Cmax/C18h≦Cmax*/C18h* preferably 1.5×Cmax/C18h≦Cmax*/C18h* and even more preferably 2.0×Cmax/C18h≦Cmax*/C18h* with
—C18h representing the plasma concentration of APIa, 18h after taking the dose,
C18h* representing the plasma concentration of APIa obtained under the same conditions as C18h, with a reference immediate-release oral pharmaceutical form, containing the same dose of APIa,
Cmax representing the maximum plasma concentration of APIa after taking the dose,
Cmax* representing the maximum plasma concentration of APIa obtained under the same conditions as Cmax, with a reference immediate-release oral pharmaceutical form, containing the same dose of APIa.

26. The drug form as claimed in claim 24 or 25, characterized in that it results in a decrease in the inter- and/or intraindividual standard deviation of the Cmax, when it is administered orally to a sample of individuals, whatever the fed state or fasting state of the individuals, compared with a pharmaceutical form for immediate release of APIa administered to this same sample of individuals, at the same dose, which makes it possible to ensure a smaller variability in effectiveness and in therapeutic safety of the pharmaceutical form.

27. The drug form as claimed in claim 26, characterized in that the factor (f) of decrease in the interindividual standard deviation of the Cmaxis defined as follows: f≧1.05; preferably, f≧1.5, and even more preferably, f is between 2.0 and 20.

28. The drug form as claimed in any one of claims 24 to 27, characterized in that it results, when it is administered orally to a sample of individuals, in a mean peak/trough modulation of the plasma profiles of the APIa less than or equal to the mean peak/trough modulation of the APIa of the same sample of individuals having received the same dose of a form for immediate release of APIa; preferably, the peak/trough modulation decrease factor g is such that: g≧1.05; preferably, g≧1.5, and even more preferably, g is between 2.5 and 20.

29. The drug form as claimed in claim 24 or 25, characterized in that it comprises microgranules for immediate release of APIa.

30. The drug form as claimed in any one of claims 24 to 29, characterized in that 70% of the APIa is released in vitro between 1 and 24 h, preferably 2 and 15 h, and more preferably 2 and 12 h.

31. The drug form as claimed in claim 30, characterized by an in vitro dissolution profile of the oral pharmaceutical form such that, for any value of the time t of between 2 h and t(70%), preferably for any value of time t of between 1 h and t(70%), the percentage of APIa dissolved is greater than or equal to 35 t/t(70%).

32. The drug form as claimed in any one of claims 24 to 29, characterized in that:
the release of the APIa is controlled by two distinct triggering mechanisms, one being based on a variation in pH and the other allowing the release of the APIa after a predetermined residence time in the stomach;
at constant pH 1.4, the dissolution profile contains a lag phase which lasts less than or equal to 7 hours, preferably less than or equal to 5 hours, and even more preferably between 1 and 5 hours,
and the passing from pH 1.4 to pH 7.0 results in a release phase which begins without any lag time.

33. The drug form as claimed in any one of claims 24 to 32, characterized in that it comprises at least two populations of microcapsules having different release profiles according to the similarity factor f2 test.

34. The drug form as claimed in any one of claims 24 to 33, characterized in that it is in the form of a single oral daily dose comprising from 1000 to 500 000 microunits containing APIa.

35. The drug form as claimed in any one of claims 24 to 34, characterized in that it is in the form of a single oral daily dose comprising from 1000 to 500 000 microcapsules for modified release of APIa.

36. The drug form as claimed in any one of claims 24 to 35, characterized in that it comprises at least one active ingredient (API) other than the APIa.

37. The drug form as claimed in any one of claims 24 to 36, characterized in that it comprises at least one suspension of microcapsules of APIa in an aqueous liquid phase which is preferably saturated or which becomes saturated with APIa on contact with the microcapsules, the coating of said microcapsules preferably having a composition corresponding to one of the following two families A′ and B′:
family A′
1A′—at least one film-forming polymer (P1) which is insoluble in the fluids of the tract, present in a proportion of 50% to 90%, preferably 50% to 80% by weight on a dry basis relative to the total mass of the coating composition, and comprising at least one water-insoluble derivative of cellulose;
2A′—at least one nitrogenous polymer (P2) present in a proportion of 2% to 25%, preferably 5% to 15% by weight on a dry basis relative to the total mass of the coating composition and consisting of at least one polyacrylamide and/or one poly-N-vinylamide and/or one poly-N-vinyllactam;
3A′—at least one plasticizer present in a proportion of 2% to 20%, preferably of 4% to 15% by weight on a dry basis relative to the total mass of the coating composition and consisting of at least one of the following compounds: glyceryl esters, phthalates, citrates, sebacates, cetyl alcohol esters, castor oil;
4A′—at least one surfactant and/or lubricant, present in a proportion of 2% to 20%, preferably of 4% to 15% by weight on a dry basis relative to the total mass of the coating composition and chosen from anionic surfactants and/or from nonionic surfactants and/or from lubricants; it being possible for said surfactant and/or lubricant to comprise just one or a mixture of the abovementioned products;
family B′
1B′—at least one film-forming polymer which is insoluble in the fluids of the gastrointestinal tract,
2B′—at least one water-soluble polymer,
3B′—at least one plasticizer,
4B′—and, optionally, at least one surfactant/lubricant, preferably selected from the group of following products:
anionic surfactants,
and/or nonionic surfactants.

38. The drug form as claimed in any one of claims 24 to 37, characterized in that it comprises at least one powder for oral suspension to be reconstituted, of microcapsules of APIa, to which the user must add the water or the liquid phase in order to reconstitute the suspension.

39. The drug form as claimed in any one of the preceding claims, characterized in that it is in the form of a sachet of microcapsule powder, of a tablet obtained from microcapsules, or of a gel capsule containing microcapsules.

40. The drug form as claimed in any one of the preceding claims, characterized in that it is free of antagonist(s) of the API or of the APIa.

41. The use of the microcapsules for modified release of APIa as defined in claim 24, and optionally of the microgranules for immediate release of APIa as defined in claim 29, for the preparation of pharmaceutical, microparticulate oral galenic forms, preferably in the form of tablets, advantageously orodispersible tablets, of powders, of gel capsules or of suspensions.

42. The use of the microcapsules for modified release of APIa as defined in claim 24, and optionally of the microgranules for immediate release of APIa as defined in claim 29, for the preparation of a therapeutically safe, microparticulate oral pharmaceutical form designed in such a way that, once said pharmaceutical form has been ingested, the microcapsules that it contains are dispersed and individualized when they reach the stomach, which allows these microcapsules to be subjected to regular and gradual gastric emptying, whether the patient had eaten or was fasting at the time the dose was taken, thus guaranteeing a release of APIa within its gastrointestinal window of bioabsorption.
